(19) United States
(12) Patent Application Publication
(10) Pub. No.: US 2024/0166730 A1
(43) Pub. Date: May 23, 2024

(54) ANTIBODY SPECIFIC TO STAPHYLOCOCCUS AUREUS, THERAPEUTIC METHOD AND DETECTION METHOD USING SAME

(71) Applicant: William R. Church, Burlington, VT (US)

(72) Inventor: William R. Church, Burlington, VT (US)

(21) Appl. No.: 17/746,660

(22) Filed: May 17, 2022

Related U.S. Application Data
(63) Continuation of application No. 16/942,077, filed on Jul. 29, 2020, now Pat. No. 11,345,744.
(60) Provisional application No. 63/055,656, filed on Jul. 23, 2020.
(60) Provisional application No. 62/844,431, filed on May 7, 2019.
(60) Provisional application No. 62/844,441, filed on May 7, 2019.

(51) Int. Cl. C07K 16/12; G01N 33/569

(52) U.S. Cl. CPC C07K 16/1271; G01N 33/56938; C07K 2317/24; G01N 2333/31

(57) ABSTRACT

We provide new monoclonal antibody inhibitors of coagulases staphylocoagulase and vWbp for treatment of S. aureus. The monoclonal antibodies are useful in targeting the SC N-terminus of SC and vWbp (respectively) and inhibiting prothrombin activation. The monoclonal antibodies are able to bind to and interfere with, modulate, and/or inhibit the binding interactions between the coagulase protein and its ligand protein prothrombin in blood and tissues. The antibodies are effective in inhibiting the activation of prothrombin.

### RELATED APPLICATIONS

The present patent document claims the benefit of the filing date under 35 U.S.C. § 119(e) of Provisional U.S. patent application Ser. Nos. 62/844,431, filed May 7, 2019, 62/844,441 filed May 7, 2019, and 63/055,656 filed Jul. 23, 2020, which are hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jul. 27, 2020, is named 163-314_SL.txt and is 45,513 bytes in size.

### BACKGROUND

Infections caused by Staphylococcus aureus (“S. aureus”) are a major causative agent of hospital and non-hospital infections. These infections cause longer hospitalization time and cost. S. aureus infections range from common, minor skin infections to blood-borne infections of the heart valves called infective endocarditis.

Infective endocarditis may be used as a model for illustrating S. aureus infection. Endocarditis is an inflammation of the endocardium, the inner layer of the heart. In some variations, it involves the heart valves. In other variations it may involve the interventricular septum, the chordae tendineae, the mural endocardium, and even surfaces of implanted medical devices such as intracardiac devices and prosthetic valves.

One characteristic of endocarditis is a lesion, which may also be referred to as a vegetation. A vegetation includes but is not limited to, a mass of platelets, fibrin, microcolonies of microorganisms, and inflammatory cells. In some variations, infective endocarditis vegetations may also include a center of granulomatus tissue, e.g., a collection of the immune cells called macrophages. Granulomatus tissue may fibrose (e.g., form excess tissue) and/or calcify.

Heart valves do not receive dedicated blood supply, which may blunt the immune response, making it difficult for immune defenses (such as white blood cells) to directly reach the valves via the bloodstream. Valves may have an increased susceptibility to infection, e.g. bacterial infection, due to (among other factors) the blunted immune response. The lack of blood supply to the valves may also decrease the effectiveness of traditional treatments, since drugs (e.g., those delivered via bloodstream) also have difficulty reaching infected valves.

S. aureus infection rates continue to increase. S. aureus acute infective endocarditis is 25-47% fatal despite antibiotic therapy. Vancomycin is a common antimicrobial treatment for infections caused by S. aureus (e.g., methicillin resistant S. aureus). Of great concern is the observation that drug resistant strains of S. aureus are rapidly evolving. The rapid spread of hypervirulent, multi-drug resistant strains of S. aureus suggest S. aureus will likely become resistant to all antibiotics and an even greater threat to public health. This threat is exacerbated due to, among other things, the reluctance of drug companies to develop new antibiotics.

### BRIEF SUMMARY

We provide monoclonal antibodies able to recognize and bind to Staphylococcus proteins including but not limited to staphylocoagulase (SC) and von Willebrand factor-binding protein (vWbp). Both SC and vWbp trigger a conformation change and induce a functional active catalytic site in the host coagulation zymogen, prothrombin (ProT). The monoclonal antibodies and fragments therefrom disclosed herein are able to bind to and interfere with, modulate, and/or inhibit the binding interactions between at least staphylocoagulase and/or von Willebrand binding protein and prothrombin, its ligand protein in blood and tissues. The disclosed antibodies, each alone and in combination, are effective in inhibiting the activation of prothrombin.

We also provide a therapeutic for use in treating a Staphylococcus infection which uses vWbp alone and/or in combination with at least one other antibody. In one example, a therapeutic for treating a Staphylococcus infection may use the anti-vWbp antibody disclosed herein and an anti-SC antibody, including but not limited to the anti-SC antibody disclosed herein.

We provide monoclonal antibodies are represented by sequences SEQ ID NO.: 5, SEQ ID NO.: 6, SEQ ID NO.: 7, SEQ ID NO.: 8. The CDRs are represented by SEQ ID NOS.: 10-15. We further provide monoclonal antibodies represented by SEQ ID NOS: 29 through 41.

Other systems, methods, features, and advantages of the disclosure will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The following description of the drawings and the various system, method, and apparatus is not intended to limit the inventive system, methods and apparatus disclosed herein to one embodiment, but rather to enable any person skilled in the art of art of antibody production to make and use the inventive system, method and monoclonal antibodies.

FIG. 1 provides a schematic of the pathogenesis of acute bacterial endocarditis.

FIG. 2 provides an exemplary representation of staphylocoagulase, and discloses SEQ ID NO: 9.

FIG. 3 provides an exemplary representation of prothrombin.

FIG. 4 provides an exemplary representation of thrombin.

FIG. 5 provides a representation of staphylocoagulase and prothrombin and their interaction resulting in the non-proteolytic formation of an active site and discloses SEQ ID NO: 9.

FIG. 6 shows immune response of three mice injected with IVTKDYSKES (SEQ ID NO: 9) conjugated to carrier protein.

FIG. 7 shows serum immune response from splenocyte donor mouse used for hybridoma production.

FIG. 8 shows binding of anti-IVTKDYSKES (SEQ ID NO: 9) antibody in hybridoma cell supernatant in ELISA.

FIG. 9 shows purified antibody designated GMA-2105 binding to conjugated- and unconjugated- IVTKDYSKES (SEQ ID NO: 9) in ELISA.

FIG. 10 shows the purified GMA-2105 antibody IgG on the SDS-PAGE assay according to a variation.

FIG. 11 shows binding of GMA-2105 F(ab) to IVTKDYSKES (SEQ ID NO: 9) coated wells in ELISA.

FIG. 12 shows a Western blot detection of staphylocoagulase in cultured bacteria of various strains using purified GMA-2105 F(ab) fragments.

FIG. 13 is a chart comparing amino acid substitutions in three mutated peptides (SEQ ID NOS 25-27, respectively, in order of appearance).

FIG. 14 shows binding of GMA-2105 to variant staphylocoagulase peptides to in solid phase competitive inhibition ELISA.

FIG. 15 shows BOPIDY-labeled staphylocoagulase fragment SC(1-325) with (S7C) titrated with GMA-2105 in solution in the presence of unlabeled SC(1-246).

FIG. 16 shows inhibition of SC(1-246)-ProT30 by GMA-2105.

FIG. 17 shows results of clotting assay demonstrating that GMA-2105 blocks staphylocoagulase activity inhibiting prothrombin activation and downstream plasma clotting.

FIG. 18 shows the effect of GMA-2105 on prothrombin activation by staphylocoagulase activity in S. aureus supernatant.

FIG. 19 shows purified GMA-2105 analyzed by size exclusion chromatography.

FIG. 20 shows dynamic light scattering of purified GMA-2105.

FIG. 21 shows detection of GMA-2105 antibody in the blood of mice 21 days post intraperitaneal administration.

FIG. 22 provides a survival curve demonstrating GMA-2105 promotes survival in a mouse model of S. aureus sepsis.

FIG. 23 provides a schematic of an exemplary chimeric GMA-2105 construct.

FIG. 24 shows binding of GMA-2105 chimeric antibody in HEK293 cell transfection supernatants to IVTKDYSKES (SEQ ID NO: 9).

FIG. 25 shows a Western blot detection of human antibody heavy chain Fc and human kappa light chain in HEK293 GMA-2105 chimeric heavy chain and light chain transfected supernatant.

FIG. 26 shows SDS-PAGE analysis of purified GMA-2105 chimera.

FIG. 27 shows purified chimeric and murine GMA-2105 antibody binding to IVTKDYSKES (SEQ ID NO: 9) in ELISA assay.

FIG. 28 shows dynamic light scattering of GMA-2105 chimera.

FIG. 29 shows BODIPY-labeled staphylocoagulase fragment SC(1-325) with (S7C) titrated with GMA-2105 chimera in solution.

FIG. 30 shows comparison of binding characteristics of murine GMA-2105 and chimeric GMA-2105.

FIG. 31 provides a theoretical model of GMA-2105 light chain SEQ ID No. 8(4-105) and GMA-2105 heavy chain SEQ ID No.: 6(4-110).

FIG. 32 provides representatives from each of the six clusters formed from the 26 lowest energy GMA-2105 models.

FIG. 33 shows a theoretical model of substitution at GMA-2105 light chain residue 46 (SEQ ID No. 8, residue 46) from alanine to leucine.

FIG. 34 shows a theoretical model of substituting an asparagine for aspartic acid at heavy chain residue 76 (SEQ ID NO.: 6, residue 76).

FIG. 35 shows a theoretical model of subsitution of a leucine for a valine at heavy chain residue 81 (SEQ ID No. 6, residue 81).

FIG. 36 shows by theoretical model the low energy models of the humanized GMA-2105 sequence.

FIG. 37 shows serum immune response from splenocyte donor mouse used for hybridoma production.

FIG. 38 shows the antibody binding curves of purified anti-vWbp GMA-2500.

FIG. 39 shows the antibody binding curves of purified anti-vWbp GMA-2510.

FIG. 40 shows the results of an activity assay of prothrombin -1 (1 nM) with vWbp (1-474)(10 nM) in the presence of increasing concentrations of GMA-2500 mAb.

FIG. 41 shows the results of an activity assay of prothrombin-1 (1 nM) with vWbp (1-474)(10 nM) in the presence of increasing concentrations of GMA-2510 mAb.

FIG. 42 shows the results of an activity assay of prothrombin-1 (1 nM) with vWbp (1-263)(10 nM) in the presence of increasing concentrations of GMA-2510 mAb.

FIG. 43 shows clotting curves for GMA-2500 and GMA-2510 versus no antibody.

FIG. 44A shows western blot of specificity of the GMA-2510 antibody.

FIG. 44B shows western blot of specificity of the GMA-2105 antibody.

FIG. 44C shows western blot of specificity of a mixture of both the GMA-2510 antibody and the 2105 antibody.

FIG. 45A shows data representing increase in turbidity as measured by absorbance change at 450 nm for mixtures of 1.5 mg/mL fibrinogen and 75 nM prothrombin complexed to vWbp-(1-263) (ProT●vWBp) complex in the absence of GMA-2510 antibody (anti-vWbp Ab; open circles), in the presence of 300 nM anti-vWbp Ab (filled circles) or 1.5 UM anti-vWbp Ab (open squares).

FIG. 45B shows data representing increase in turbidity as measured by absorbance change at 450 nm for mixtures of 1.5 mg/mL fibrinogen and for 15 nM prothrombin complexed to SC-(1-325) (ProT●SC) are shown in the absence of GMA-2105 antibody (anti-SC Ab), in the presence of 50 nM anti-SC ab (filled circles) or 250 nM (open squares) anti-SC ab.

FIG. 46A shows in vivo FMT/CT images of S. aureus endocarditis in mice after injection of DAB-VT680XL treated with isotype control antibody.

FIG. 46B shows FIG. 46A shows in vivo FMT/CT images of S. aureus endocarditis in mice after injection of DAB-VT680XL treated with antibodies neutralizing SC and vWbp.

FIG. 47 shows Kaplan-Meier survival curves of S. aureus endocarditis mice treated with isotype control antibody or combination therapy with anti-SC and anti-vWbp antibodies.

FIG. 48 shows intravital microscopy of femoral S. aureus vegetation 24 hours after intravenous injection of S. aureus^{RFP+} and combination treatment with both anti-SC and anti-vWbp.

FIG. 49 shows intravital microscopy of femoral S. aureus vegetation 24 hours after intravenous injection of S. aureus^{RFP+} isotype control antibody.

### DETAILED DESCRIPTION

S. aureus, is a highly adaptive human pathogen, causing recurrent skin and soft tissue infections by evading the immune system. S. aureus colonizes the nose and skin of 20-30% of the human population. Healthy individuals with intact skin and mucosal barriers may harbor S. aureus with no adverse result. However, when the integrity of the skin and mucosa are breached, S. aureus can invade and enter the tissue and blood stream and potentially cause injury.

Pathologies associated with S. aureus colonization include but are not limited to meningitis, sepsis, pneumonia. Pathologies also include endocarditis and septic arthritis in high risk populations, such as infants, immunocompromised adults, and intravenous drug users. The presence of foreign materials in the body, including intravenous catheters, greatly increases the risk of developing S. aureus-induced endocarditis. For example, catheters may become coated with fibrinogen and fibronectin, to which the bacterium can easily adhere.

Therapy of staphylococcus infections, including but not limited to, methicillin-resistant S. aureus (MRSA) infections, is complicated by the fact that the organisms have evolved resistance to commonly used therapeutics and quickly develop immunity to new therapeutics. For example, despite antibiotic therapy, MRSA infections are associated with a poor outcome. It is estimated that only 5% of S. aureus isolates are susceptible to penicillin treatment. Another feature of staphylococcal infections is its reoccurrence rate. Further complicating treatment indications, clinical and experimental observations suggest that infections with S. aureus do not generate protective immune response.

The immune invasion strategies of S. aureus allow it to survive in the blood. Through the blood and/or subepidermal tissues, S. aureus encounters and escapes the host innate immune defenses. S. aureus displays cell-surface proteins and secreted virulence factors, including the procoagulants staphylocoagulase and von Willebrand factor-binding protein which allow it to compromise the effectiveness of both the innate and adaptive immune responses. Some examples of how S. aureus evades the host immune system includes, but is not limited to, invading endothelial cells and neutrophils, inhibiting the complement system, and subverting the coagulation system for infective purposes.

Acute infective endocarditis (AIE) is one of the illnesses caused by S. aureus. Other illnesses include metastatic infections migrating to bones/joints, spleen, kidneys, liver, and lungs; sepsis; toxic shock syndrome; and pneumonia (among others). S. aureus recruits new virulence factors and antibiotic resistance encoded by mobile genetic elements (MGEs) from other strains or different bacterial species. The emergence of MRSA is attributed to MGE transfer, as is the ongoing global epidemic of the hypervirulent USA300 strain of community associated-MRSA (CA-MRSA).

Antibiotic resistance of CA-MRSA is leading to a reliance on vancomycin for treatment of severe infections. However, S. aureus has also developed resistance to this antibiotic. Partially vancomycin-resistant S. aureus with thickened cell walls have been described and clinical isolates carrying the vanA gene complex have appeared in the United States.

The adaptability of S. aureus and the rapid spread of hypervirulent and multidrug-resistant strains supports the prediction that S. aureus will soon become resistant to all available antibiotics. This huge public health problem will be associated with increased morbidity and mortality based on the propensity of S. aureus to cause many potentially lethal infections. The elderly are particularly susceptible and as the U.S. population ages there will be an increase in these infections.

The public health problem is exacerbated by the lack of interest of pharmaceutical companies in developing new antibiotics. This derives from the rapid emergence of antibiotic resistance that reduces the market life of the drugs and low profit due to the short treatment time. Attempts to create staphylococcal vaccines have uniformly failed, as demonstrated by clinical trial data.

To begin to address the public health threat posed by S. aureus we disclose new monoclonal antibodies as mechanism-targeted inhibitors of S. aureus procoagulants, including SC and vWbp. We also disclose a novel therapeutic which may include an anti-vWbp antibody, or fraction thereof, alone or combined with a second antibody or fraction thereof, including but not limited to an anti-SC antibody or fragment thereof.

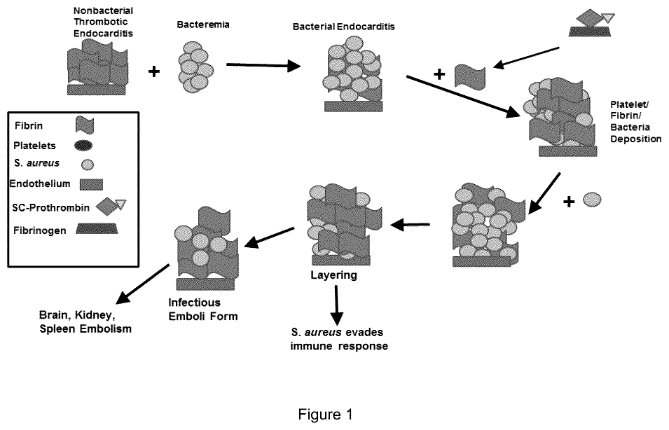

Acute infectious endocarditis (AIE) pathogenesis of S. aureus has at least two initiating events (FIG. 1). First, injury to the endothelium covering the heart valves. In one example, injury may result from turbulent flow due to a congenital or acquired defect. Injury may also result from the presence of intravascular catheters, intravenous drug use, or physiological stress from hypersensitivity states, hormonal changes, or exposure to high altitude.

Injury may initiate coagulation and formation of a sterile thrombus composed of, e.g., fibrin and platelets. Coagulation may be initiated due to exposure of blood to, among others, tissue factor. Exposure of blood to tissue factor triggers activation of blood coagulation. Activation of blood coagulation may result in formation of a sterile thrombus. The sterile thrombus may be composed of, among other factors, activated platelets and fibrin (e.g., thrombin-generated fibrin).

A compromised system may permit bacteria to enter the bloodstream and adhere to the sterile thrombis, for example, by binding fibrinogen and fibronectin. S. aureus is among the most common bacterial pathogens. S. aureus expresses cell surface components, for example but not limited to adhesins. S. aureus cell surface components include but are not limited to wall-bound adhesins that bind fibrin(ogen), e.g., clumping factor A (CIfA) and other microbial surface components. The various cell surface components recognize adhesive matrix molecules that mediate binding of the bacteria to the site of vascular injury.

The most aggressive S. aureus strains in AIE secrete SC and also vWbp, which bind to host prothrombin, abbreviated “II.” The active (active is designated “*”) SC.prothrombin* (SC●II*) and vWbp●II* complexes bind host fibrinogen as their substrate and convert it into fibrin. Deposition of fibrin propagates the formation of platelet-fibrin-bacteria vegetations, characteristic of AIE, at the site of vascular injury.

Vegetations grow by layering of more bacteria onto fibrin, and fibrin generation by the SC●II* and vWbp●II* catalytic complexes (FIG. 1). Fibrin layers on vegetations and protects the bacteria from clearance by immune cells and killing by antibiotics. Turbulent flow nearby vegetations propagates endothelial damage across heart valves, ultimately leading to valvular dysfunction due to tissue damage and heart failure. Large vegetations produce infectious emboli that spread through the bloodstream to distant sites where they form abscesses in the brain, spleen, and kidneys.

Embolism to the brain is common in S. aureus AIE occurring in 30% of patients and results in ischemic or hemorrhagic stroke, which is often fatal. Mortality from AIE is very high at 25-47%, despite aggressive antibiotic treatment. Clearly, adjunctive therapy is sorely needed, which we begin to address here in the form of mechanism-based monoclonal antibodies.

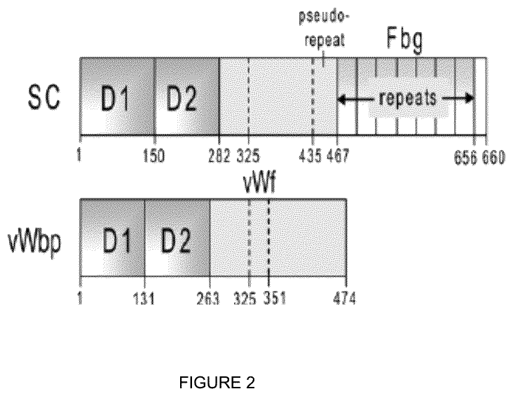

FIG. 2 provides a schematic diagram of SC and vWbp, the two S. aureus virulence factors responsible for thrombin activation and subsequent fibrin generation and clotting. The proteins have a similar N-terminus nut differ at the C termini. SC has a fibrinogen binding region. Von Willebrand factor binding protein binds von Willebrand factor. The homologous N termini are responsible for the activation of prothrombin.

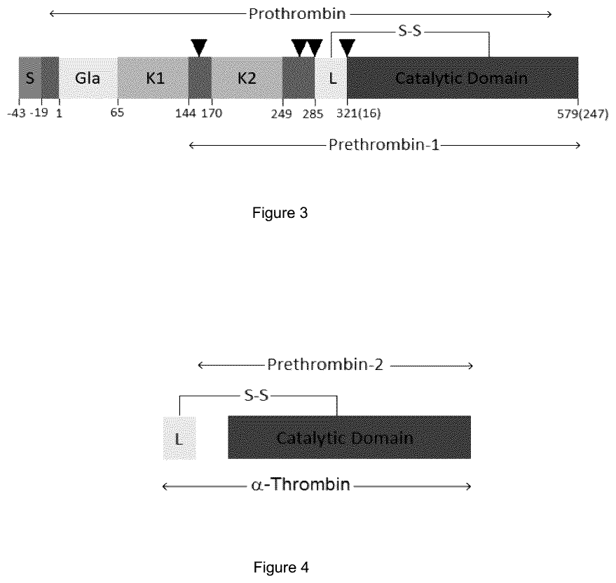

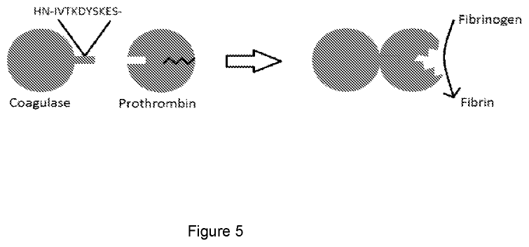

FIG. 3 provides a schematic diagram of prothrombin. FIG. 4 provides a schematic diagram of thrombin. FIG. 5 is a diagram of the interaction of staphylocoagulase with prothrombin resulting in the non-proteolytic formation of an active site. The N-terminal of staphylocoagulase forms a salt bridge with Asp 194 (chymostrypsin numbering) in the prothrombin activation site resulting in a conformational change in prothrombin which nonproteolytically stabilizes the thrombin active site. Fibrinogen can then be cleaved to fibrin by thrombin, resulting in the formation of a clot. (See FIG. 1).

The SC(1-325)●Pre 2* structure shows the N-terminus of SC (IIe¹-Val²) inserted into the IIe¹⁶ (chymotrypsinogen numbering) N-terminal binding cleft of the prothrombin fragment designated Pre 2 where it forms the critical salt-bridge with Asp194 that triggers non-proteolytic activation of Pre 2 within the complex. Based on the SC(1-325) structure, a family of staphylococcal and streptococcal homologs named zymogen activator and adhesion proteins (ZAAPs) was postulated, including vWbp. This prompted the initial identification of vWbp as a coagulase based on plasma clotting experiments. Prior studies demonstrated vWbp activates prothrombin by binding and expressing a thrombin active site. The complex is able to cleave substrate fibrinogen.

Recognizing the problems inherent in existing treatments, such as but not limited to, rapid resistance including emerging Vancomycin resistance and off-target events associated with anticoagulants, we disclose a method, system, and monoclonal antibody therapeutic which enables the diagnosis and treatment of S. aureus infection in a specific and accurate manner by using SC and vWbp-specific monoclonal antibodies, alone or in combination with other antibodies. In one example, vWbp-specific antibodies are used in combination with staphylocoagulase-specific antibodies.

Accordingly, we provide monoclonal antibodies that can bind to the staphylocoagulase protein and we also provide monoclonal antibodies that can bind to the vWbp from S. aureus, or certain subregions thereof, with high affinity, preventing the S. aureus SC and/or vWbp from forming an active pro-thrombin-bacterial cofactor complex. The monoclonal antibodies disclosed herein, thereby prevent the formation of fibrin, which prevents S. aureus' ability to create the vegetations used to protect the bacteria from clearance by immune cells and killing by antibiotics.

We demonstrate herein the effectiveness of anti-staphylocoagulase and/or vWbp monoclonal antibodies to prevent fibrin formation (as demonstrated by clotting data, among other things) and to successfully treat S. aureus infections in an animal model. We further demonstrate herein the effectiveness of anti-vWbp monoclonal antibodies in combination with anti-staphylocoagulase antibodies to prevent fibrin formation (as demonstrated by clotting data, among other things) and to successfully improve survival rates from S. aureus infections in an animal model.

The results of our experiments herein were surprising in light of the fact that the role of staphylocoagulase and/or vWbp as a virulence factor in S. aureus infection has been in doubt due to conflicting results in a rat model of endocarditis.

Fully developed S. aureus abscesses exhibit a defined structure with the bacteria in the center, bordered by fibrin-rich barriers that protect the bacteria from immune cells and antibiotics. The role of staphylocoagulase has been in doubt also in light of research demonstrating that S. aureus lacking either the staphylocoagulase or vWbp gene could form abscesses.

The use of anticoagulants, which have the potential to produce lethal bleeding, to treat AIE has been debated. Given the complexity of thrombin regulation through its two exosites that bind protein substrates, inhibitors, and regulatory macromolecules, and its many procoagulant and anticoagulant roles, systemic inhibition of thrombin (via anti-coagulants) in an attempt to inhibit SC●II* and vWbp●II* may lead to off-target events with adverse consequences.

Comparison of 35 patients with S. aureus AIE to those with native valve AIE, of which none were taking anticoagulants, to 21 with prosthetic valve AIE 90% of which were taking anticoagulants, found that the groups had similar incidence of neurologic embolisms, and the mortality in the anticoagulant group was 71% compared to 37% in the no anticoagulant group. More of those patients had brain hemorrhages. An old study found the opposite outcome for a small group of patients with prosthetic valves; i.e., mortality was slightly greater (57% vs. 47%) if anticoagulant therapy was stopped.

Our novel monoclonal antibodies targeting, in one instance staphylocoagulase and in another instance vWbp, avoids the off-target events experienced with anti-coagulants. The SC- and/or vWbp monoclonal antibodies inhibit fibrin formation by targeting the staphylocoagulase and/or vWbp protein specifically. It therefore has a localized effect at the site of S. aureus infections and does not have system-wide consequences. We demonstrate that the novel monoclonal antibody has the ability to improve survival rates in the mouse model alone and/or in combination with the anti-staphylocoagulase antibody disclosed herein.

The mouse models for blood coagulation are similar to the human coagulation system. Emeis, et al., A guide to murine coagulation factor structure, function, assays and genetic alterations, J. Thromb. Haemost. 2007; 5:670-9, incorporated herein by reference in its entirety. Numerous therapeutics directed at human coagulation systems (clotting, platelets, fibrinolysis) are similarly effective at comparable doses in mice. This suggests that targeting staphylocoagulase and/or vWbp with an antibody might give physiologic and pharmacologic data that is reflective of what happens in humans.

Targeting S. aureus via the host hemostatic response is a novel therapeutic approach to treating S. aureus infection. The mechanistic details of prothrombin activation are well known. (See, e.g., Krishnaswamy, S., The transition of prothrombin to thrombin, J. Thromb. Haemost, 2013; 11:256-76, incorporated herein by reference in its entirety.) The novel approach involves using a monoclonal antibody to block S. aureus' ability to form a fibrin mesh, preventing the course of disease. (The course of disease is illustrated in FIG. 1). A therapeutic monoclonal antibody provides advantages over vaccines. In contrast to a vaccine that requires the development of an immune response which may take several days or months, the therapeutic monoclonal antibody can be used in acute infections and can also offer prophylactic protection before an infection. We demonstrate that our novel anti-staphylocoagulase and/or anti-vWbp monoclonal antibodies prevent fibrin formation.

Accordingly, we provide monoclonal antibodies that can bind to the SC and/or vWbp protein from S. aureus, or certain subregions thereof, with high affinity and which can thus be useful in methods to treat, prevent, or diagnose staphylococcal infections.

We provide a therapeutic antigen binding protein which recognizes an epitope of the SC protein from S. aureus that contains residues 1-12 from the N-terminal SC.

We also provide a therapeutic antigen binding protein which recognizes an epitope of the vWbp protein from S. aureus that contains residues 1-12 from the N-terminal vWbp.

We also provide a therapeutic antigen binding protein which recognizes an epitope of the SC protein from S. aureus that contains one or more of residues 1, 2, 3, 4, 5, 6, 7, 8, 9, and/or 10 and/or 1-3, 1-4, 1-5, 1-6, 1-7, 1-8, 1-9,1 or 1-10 of the SC protein.

We also provide a therapeutic antigen binding protein which recognizes an epitope of the vWbp protein from S. aureus that contains one or more of residues 1, 2, 3, 4, 5, 6, 7, 8, 9, 10, 11 and/or 12 and/or 1-3, 1-4, 1-5, 1-6, 1-7, 1-8, 1-9,1-10, 1-11 or 1-12 of the vWbp protein.

In a variation, we also provide a therapeutic antigen binding protein which requires residues 1 and 2 of the SC peptide sequence: IVTKDYSKES (SEQ ID NO: 9).

In a variation, we also provide a therapeutic antigen binding protein which requires residues 1 and 2 of the vWbp peptide sequence: VVSGEKNPYVSK (SEQ ID NO: 28).

In a variation, the therapeutic antigen binding protein is an antibody or antigen binding fragment and/or derivative thereof.

We also provide monoclonal antibodies which are able to bind at least one of the S. aureus SC and/or vWbp peptides and/or peptides which are generated from the S. aureus SC and/or vWbp peptides or a subregion thereof and which are useful in methods to treat, prevent, or diagnose staphylococcal infections. In one example, we provide monoclonal antibodies generated from the N-terminal SC, in another example, we provide monoclonal antibodies generated from the SC(1-12). In another example, we provide monoclonal antibodies generated from SC(1-10). In another example, we provide monoclonal antibodies generated from a peptide comprising amino acids IVTKDYSKES (SEQ ID No. 9) and homologs and/or degenerations thereof.

In another example, we provide monoclonal antibodies generated from the N-terminal vWbp, in another example, we provide monoclonal antibodies generated from the vWbp N-terminal (1-12). In another example, we provide monoclonal antibodies generated from vWbp N-terminal (1-10). In another example, we provide monoclonal antibodies generated from a peptide comprising amino acids VVSGEKNPYVSK (SEQ ID No. 28) and homologs and/or degenerations thereof.

We also provide monoclonal antibodies to the SC protein which can be useful in inhibiting the SC-II activation.

We also provide monoclonal antibodies capable of binding vWbp, which may be helpful in inhibiting the vWbp-II activation.

We also provide a therapeutic agent having multiple monoclonal antibodies or fragments thereof. As a non-limiting example, a therapeutic may have an anti-vWbp antibody combined with a second antibody. In a further non-limiting example, a therapeutic may have an anti-vWbp antibody combined with an anti-SC antibody.

We also provide monoclonal antibodies able to recognize and bind to Staphylocoagulase protein (SEQ ID No. 2) and other proteins presenting sequences homologous to Staphylocoagulase including vWbp (SEQ ID No. 4), and other bacteria possessing Staphylocoagulase, vWbp or sequences homologous thereto.

We also provide monoclonal antibodies to recognize and bind to proteins encoded by staphylocoagulase nucleotide sequences (SEQ ID No. 1) and other proteins encoded by sequences homologous to staphylocoagulase including vWbp (SEQ ID No. 3). The monoclonal antibodies may also recognize and bind to proteins of bacteria possessing staphylocoagulase, vWbp or sequences homologous to staphylocoagulase, vWbp.

We also provide monoclonal antibodies able to recognize and bind to peptide sequence IVTKDYSKES (SEQ ID NO: 9), Staphylocoagulase peptide sequence VVSGEKNPYVSK (SEQ ID NO: 28), and other proteins presenting sequences homologous to peptide sequence IVTKDYSKES (SEQ ID NO: 9), and Staphylocoagulase peptide sequence VVSGEKNPYVSK (SEQ ID NO: 28).

We also provide a method which enables the treatment of S. aureus in a specific and accurate manner by using SC specific monoclonal antibodies generated using SC(1-10) peptide sequence (SEQ ID No. 9 and amino acids 1-10 of SEQ ID NO.: 2).

We also provide a method which enables the treatment of S. aureus in a specific and accurate manner by using vWbp specific monoclonal antibodies generated using vWbp(1-12) peptide sequence (SEQ ID No. 28 and amino acids 1-12 of SEQ ID NO.: 4).

We provide an antibody specifically binding to a polypeptide comprising a SC, wherein the monoclonal antibody was generated using a peptide comprising the amino acid sequence IVTKDYSKES (SEQ ID No. 9).

We provide an antibody specifically binding to a polypeptide comprising a vWbp, wherein the monoclonal antibody was generated using a peptide comprising the amino acid sequence VVSGEKNPYVSK (SEQ ID No. 28).

We also provide at least one antibody capable of binding SC(1-10) and/or vWbp (1-12) and inhibiting the binding of SC and/or vWbp to prothrombin.

We also provide an antibody capable of binding SC(1-12) inhibiting the activation of prothrombin³⁰ (a non-cleavable form of prothrombin) by SC(1-246) with 0.9 nM affinity.

We also provide an antibody capable of binding to SC which has an affinity constant (KD) for SC of between 0.1 nm and 10 nM, specifically 2 nM for binding of 1.6 mol Bodipy-SC/mol mAb.

We also provide a method of treating an individual infected with S. aureus by injecting the infected individual with monoclonal antibody against at least one of SC and/or vWbp.

We also provide an antigen binding protein, including but not limited to a therapeutic antigen binding protein, such as an antibody or antigen binding fragment and/or derivative thereof, which binds S. aureus SC and which comprises the heavy chain variable region amino acid sequence of SEQ ID NO.: 6 and the antibody light chain variable region of SEQ ID NO.: 8.

We also provide an antigen binding protein, including but not limited to a therapeutic antigen binding protein, such as an antibody or antigen binding fragment and/or derivative thereof, which binds S. aureus vWbp and which comprises the heavy chain variable region amino acid sequence of SEQ ID NO.: 29 and the antibody light chain variable region of SEQ ID NO.: 31.

We also provide an antigen binding protein, including but not limited to a therapeutic antigen binding protein, such as an antibody or antigen binding fragment and/or derivative thereof, which binds S. aureus vWbp and which comprises the heavy chain variable region amino acid sequence of SEQ ID NO.: 35 and the antibody light chain variable region of SEQ ID NO.: 37.

We also provide an antigen binding protein, including but not limited to a therapeutic antigen binding protein, such as an antibody or antigen binding fragment and/or derivative thereof, which binds SC and which comprises the following CDRs:

CDRL1:
QNVDIY
(residues 27-32 of SEQ ID No. 8 and
SEQ ID No: 10)
CDRL2:
SAS
(residues 50-52 of SEQ ID No. 8 and
SEQ ID NO.: 11)
CDRL3:
QQYNNYPYT
(residues 89-97 of SEQ ID No. 8 and
SEQ ID No: 12)
CDRH1:
GFTFSDAW
(residues 26-33 of SEQ ID No. 6 and
SEQ ID NO: 13)
CDRH2:
IRTKANNHAT
(residues 51-60 of SEQ ID No. 6 and
SEQ ID NO: 14)
CDRH3:
CTNVYYGNNDVKDY
(residues 98-111 of SEQ ID No. 6 and
SEQ ID NO: 15).

We also provide an antigen binding protein, including but not limited to a therapeutic antigen binding protein, such as an antibody or antigen binding fragment and/or derivative thereof, which binds vWbp and which comprises the following CDRs:

CDRH1:
GYSFTSYWIH
(Residues 26-35 of SEQ ID NO. 29)
CDRH2:
AISPGNSDTNYNQNFKG
(Residues 50-66 of SEQ ID NO. 29)
CDRH3:
ATGSHY
(Residues 97-102 of SEQ ID NO. 29)
CDRL1:
QSLLDSDGTTY
(Residues 27-37 of SEQ ID NO. 31)
CDRL2:
LVS
(Residues 55-57 of SEQ ID NO. 31)
CDRL3:
WQGTHFPRT
(Residues 94-102 of SEQ ID NO. 31).

We also provide an antigen binding protein, including but not limited to a therapeutic antigen binding protein, such as an antibody or antigen binding fragment and/or derivative thereof, which binds vWbp and which comprises the following CDRs:

CDRH1:
GFTFTNYF
(Residues 26-33 of SEQ ID NO. 35)
CDRH2:
IYTGNGDT
(Residues 51-58 of SEQ ID NO. 35)
CDRH3:
NYAMDH
(Residues 97-102 of SEQ ID NO. 35)
CDRL1:
QSLLDSDGKTY
(Residues 27-37 of SEQ ID NO. 37)
CDRL2:
LVS
(Residues 55-57 of SEQ ID NO. 37)
CDRL3:
WQGTHFPRT
(Residues 94-102 of SEQ ID NO. 37)

We also provide an antigen binding protein, such as an antibody or antigen binding fragment thereof which specifically binds SC and comprises CDR's which are variants of the sequences set forth in SEQ ID NOS.: 10 through 15 and 28 through 41.

A variant includes a partial alteration of the CDR, heavy chain and/or light chain amino acid sequence by deletion or substitution of one to several amino acids of the CDR, heavy chain and/or light chain, or by addition or insertion of one to several amino acids to the CDR, heavy chain, and/or light chain, or by a combination thereof. The variant may contain 1, 2, 3, 4, 5, or 6 amino acid substitutions, additions or deletions in the amino acid sequence of the CDR, heavy chain, and/or light chain sequence. The substitutions in amino acid residues may be conservative substitutions, for example, substituting one hydrophobic amino acid for an alternative hydrophobic amino acid.

Antigen binding proteins which are variants of the CDR, heavy chain, and/or light chain will have the same or similar functional properties to those comprising the CDR, heavy chain, and/or light chain described herein. Therefore, antigen binding proteins which comprise a variant CDR will bind to the same target protein or epitope with the same or similar binding affinity to the CDR, heavy chain, and/or light chain described herein.

In a variation there is provided a humanized or chimeric antibody comprising the following CDRs of GMA-2500:

CDRH1:
GYSFTSYWIH
(Residues 26-35 of SEQ ID NO. 29)
CDRH2:
AISPGNSDTNYNQNFKG
(Residues 50-66 of SEQ ID NO. 29)
CDRH3:
ATGSHY
(Residues 97-102 of SEQ ID NO. 29)
CDRL1:
QSLLDSDGTTY
(Residues 27-37 of SEQ ID NO. 31)
CDRL2:
LVS
(Residues 55-57 of SEQ ID NO. 31).
CDRL3:
WQGTHFPRT
(Residues 94-102 of SEQ ID NO. 31).

For example, a chimeric antibody may comprise the variable regions of the GMA-2500 antibody, namely SEQ ID No: 29 (VH) and SEQ ID No. 31 (VL) in with the constant regions from another species, such as a human.

In a variation there is provided a humanized or chimeric antibody comprising the following CDRs of GMA-2510:

CDRH1:
GFTFTNYF
(Residues 26-33 of SEQ ID NO. 35)
CDRH2:
IYTGNGDT
(Residues 51-58 of SEQ ID NO. 35)
CDRH3:
NYAMDH
(Residues 97-102 of SEQ ID NO. 35)
CDRL1:
QSLLDSDGKTY
(Residues 27-37 of SEQ ID NO. 37)
CDRL2:
LVS
(Residues 55-57 of SEQ ID NO. 37)
CDRL3:
WQGTHFPRT
(Residues 94-102 of SEQ ID NO. 37)

For example, a chimeric antibody may comprise the variable regions of the GMA-2105 antibody, namely SEQ ID No: 35 (VH) and SEQ ID No. 37 (VL) in with the constant regions from another species, such as a human.

In further variations we provide antibody derived proteins with the ability to bind to two or more different epitopes. In an example, an antibody derived protein may contain a combination of variable regions. In an example, the antibody derived protein may contain variable regions specific for binding SC combined with variable regions specific for binding vWbp.

For example, a binding protein or fragment thereof which binds in one region an epitope of vWbp that is within SEQ ID. No: 4 and which comprises the following CDRs:

CDRH1:
GFTFTNYF
(Residues 26-33 of SEQ ID NO. 35);
CDRH2:
IYTGNGDT
(Residues 51-58 of SEQ ID NO. 35);
and
CDRH3:
NYAMDH
(Residues 97-102 of SEQ ID NO. 35);
In addition to at least one of either:
CDRL1:
QSLLDSDGKTY
(Residues 27-37 of SEQ ID NO. 37);
CDRL2:
LVS
(Residues 55-57 of SEQ ID NO. 37);
and
CDRL3:
WQGTHFPRT
(Residues 94-102 of SEQ ID NO. 37);
Or
CDRL1:
QSLLDSDGTTY
(Residues 27-37 of SEQ ID NO. 31);
CDRL2:
LVS
(Residues 55-57 of SEQ ID NO. 31);
and
CDRL3:
WQGTHFPRT
(Residues 94-102 of SEQ ID NO. 31).

The binding protein capable of binding with two specificities may binds an epitope of SC that is within SEQ ID. No: 2 and which comprises the following CDRs:

CDRL1:
QNVDIY
(residues 27-32 of SEQ ID No. 8);
CDRL2:
SAS
(residues 50-52 of SEQ ID No. 8)
CDRL3:
QQYNNYPYT
(residues 89-97 of SEQ ID No. 8)
CDRH1:
GFTFSDAW
(residues 26-33 of SEQ ID No. 6)
CDRH2:
IRTKANNHAT
(residues 51-60 of SEQ ID No. 6)
CDRH3:
CTNVYYGNNDVKDY
(residues 98-111 of SEQ ID No. 6).

In another example we provide a binding protein or fragment thereof which binds an epitope of vWbp that is within SEQ ID. No: 4 and which comprises the following CDRs:

CDRH1:
GYSFTSYWIH
(Residues 26-35 of SEQ ID NO. 29);
CDRH2:
AISPGNSDTNYNQNFKG
(Residues 50-66 of SEQ ID NO 29);
and
CDRH3:
ATGSHY
(Residues 97-102 of SEQ ID NO. 29).

In addition to at a light chain that is at least one of either:

CDRL1:
QSLLDSDGKTY
(Residues 27-37 of SEQ ID NO. 37);
CDRL2:
LVS
(Residues 55-57 of SEQ ID NO. 37);
and
CDRL3:
WQGTHFPRT
(Residues 94-102 of SEQ ID NO. 37);
Or
CDRL1:
QSLLDSDGTTY
(Residues 27-37 of SEQ ID NO. 31);
CDRL2:
LVS
(Residues 55-57 of SEQ ID NO. 31);
and
CDRL3:
WQGTHFPRT
(Residues 94-102 of SEQ ID NO. 31).

In an example, we provide a therapeutic agent that contains a monoclonal antibody or fragment thereof which binds an epitope of vWbp that is within SEQ ID. No: 4 and which has the following CDRs:

CDRH1:
GFTFTNYF
(Residues 26-33 of SEQ ID NO. 35);
CDRH2:
IYTGNGDT
(Residues 51-58 of SEQ ID NO. 35);
and
CDRH3:
NYAMDH
(Residues 97-102 of SEQ ID NO. 35);
CDRL1:
QSLLDSDGKTY
(Residues 27-37 of SEQ ID NO. 37);
CDRL2:
LVS
(Residues 55-57 of SEQ ID NO. 37);
and
CDRL3:
WQGTHFPRT
(Residues 94-102 of SEQ ID NO. 37);

monoclonal antibody or fragment thereof which binds an epitope of SC that is within SEQ ID. No: 2 and which comprises the following CDRs:

CDRL1:
QNVDIY
(residues 27-32 of SEQ ID No. 8);
CDRL2:
SAS
(residues 50-52 of SEQ ID No. 8)
CDRL3:
QQYNNYPYT
(residues 89-97 of SEQ ID No. 8)
CDRH1:
GFTFSDAW
(residues 26-33 of SEQ ID No. 6)
CDRH2:
IRTKANNHAT
(residues 51-60 of SEQ ID No. 6)
CDRH3:
CTNVYYGNNDVKDY
(residues 98-111 of SEQ ID No. 6).

We also provide a binding fragment with a V_{H} region having at least 95% identity with SEQ ID NO: 35 and a V_{L} region at least 95% identity with SEQ ID NO: 37.

We also provide a binding fragment with a V_{H} region having at least 95% identity with SEQ ID NO: 35 and a V_{L} region at least 95% identity with SEQ ID NO: 31.

We also provide a binding fragment with a V_{H} region having at least 95% identity with SEQ ID NO: 29 and a V_{L} region at least 95% identity with SEQ ID NO: 37.

We also provide a binding fragment with a V_{H} region having at least 95% identity with SEQ ID NO: 29 and a Vi region at least 95% identity with SEQ ID NO: 31.

We also provide a binding fragment combining any of the following segments, individually or in combinations of one or more:

CDRL1:
QNVDIY
(residues 27-32 of SEQ ID No. 8);
CDRL2:
SAS
(residues 50-52 of SEQ ID No. 8);
CDRL3:
QQYNNYPYT
(residues 89-97 of SEQ ID No. 8);
CDRH1:
GFTFSDAW
(residues 26-33 of SEQ ID No. 6);
CDRH2:
IRTKANNHAT
(residues 51-60 of SEQ ID No. 6);
CDRH3:
CTNVYYGNNDVKDY
(residues 98-111 of SEQ ID No. 6);
CDRH1:
GFTFTNYF
(Residues 26-33 of SEQ ID NO. 35);
CDRH2:
IYTGNGDT
(Residues 51-58 of SEQ ID NO. 35);
CDRH3:
NYAMDH
(Residues 97-102 of SEQ ID NO. 35);
CDRL1:
QSLLDSDGKTY
(Residues 27-37 of SEQ ID NO. 37);
CDRL2:
LVS
(Residues 55-57 of SEQ ID NO. 37);
CDRL3:
WQGTHFPRT
(Residues 94-102 of SEQ ID NO. 37);
CDRH1:
GYSFTSYWIH
(Residues 26-35 of SEQ ID NO. 29);
CDRH2:
AISPGNSDTNYNQNFKG
(Residues 50-66 of SEQ ID NO. 29);
CDRH3:
ATGSHY
(Residues 97-102 of SEQ ID NO. 29);
CDRL1:
QSLLDSDGTTY
(Residues 27-37 of SEQ ID NO. 31);
CDRL2:
LVS
(Residues 55-57 of SEQ ID NO. 31);
and/or
CDRL3:
WQGTHFPRT
(Residues 94-102 of SEQ ID NO. 31.

Method of Generating Monoclonal Antibodies

We provide monoclonal antibodies that can recognize and bind to, among other proteins, staphylocoagulase and/or vWbp, and homologs thereof. In one method, the antibodies to SC are raised against a synthetic peptide comprising the sequence IVTKDYSKES (SEQ ID NO.: 9) and the antibodies to vWbp are raised against a synthetic peptide comprising the sequence VVSGEKNPYVSK (SEQ ID NO.: 28).

The synthetic peptide was used to generate a panel of murine monoclonal antibodies. Monoclonal antibodies recognizing staphylocoagulase and/or vWbp may also be raised from peptides comprising expressed and purified staphylocoagulase and/or vWbp, and/or synthetic peptides raised from other subregions or larger immunogenic regions of the staphylocoagulase and/or vWbp protein.

Antibodies may be obtained in conventional ways including steps of introducing the antigen, subregions, peptides, or degenerate versions thereof, into a host animal, followed by isolation of the antibody-producing spleen cells and formation of a suitable hybridoma.

An inhibitory mouse anti-staphylocoagulase antibody designated GMA-2105 was produced against a peptide IVTKDYSKES (SEQ ID NO.: 9), representing residues 1-10 of staphylocoagulase (SEQ ID NO.: 2), with the achieved goal of blocking activation of prothrombin by binding to the staphylocoagulase sequence that inserts into prothrombin.

Inhibitory mouse anti-vWbp antibodies designated GMA-2500 and GMA-2510 were produced against a peptide VVSGEKNPYVSK (SEQ ID NO.: 28), representing residues 1-12 of vWbp (SEQ ID NO.: 4), with the achieved goal of blocking activation of prothrombin by binding to the vWbp sequence.

Example Method of Generating Anti- Staphylocoagulase and/or anti-vWbp Monoclonal Antibody.

Female Balb/c mice approximately 6-8 weeks old received an initial intraperitoneal (ip) injection of either 100 μg staphylocoagulase peptide (sequence IVTKDYSKES, SEQ ID NO.: 9) or vWbp peptide (VVSGEKNPYVSK, SEQ ID NO.: 28) conjugated with-keyhole limpet hemocyanin (KLH) and emulsified in complete Freund's adjuvant on day 0. Booster injections of 50 μg of the above peptide conjugate emulsified in incomplete Freund's were given on days 10, 20 and 30.

Two additional 50 μg injections (peptide-KLH conjugate in incomplete Freunds adjuvant) were given on days 61 and 111. A serum sample from each mouse was collected on Day 122 and the antibody titer measured by solid-phase Enzyme Linked Immunosorbant Assay (ELISA) using the staphylocoagulase and/or vWbp peptide conjugated to ovalbumin coated on 96-well ELISA plates at 2 ug/ml. Uncoated areas of the wells were blocked with albumin from bovine serum (BSA). The wells were then incubated with dilutions of the mouse serum to test whether the serum contained antibodies to the staphylocoagulase and/or vWbp peptide. Bound antibodies were detected with anti-mouse IgG secondary antibody conjugated to horseradish peroxidase. The plates were then incubated with o-phenylenediamine (OPD) substrate which undergoes a color change in the presence of HRP the intensity of which can be measured using absorption at 490 nm wavelength in a microplate reader.

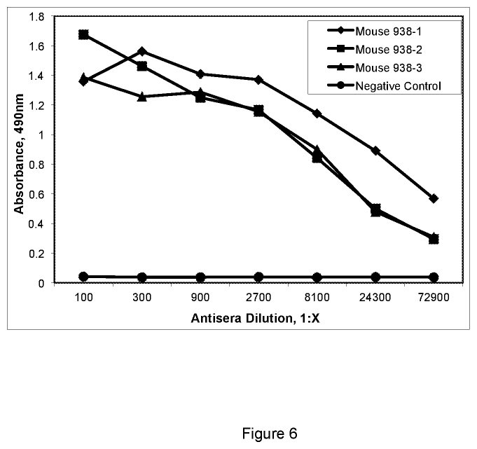

FIG. 6 provides the results of the ELISA demonstrating the presence of anti-staphylocoagulase antibodies in mouse serum of mice (designated 938-1, 938-2, and 938-3 on FIG. 6) injected with the staphylocoagulase peptide SC(1-10) (SEQ ID NO.: 9). Serum from an irrelevant mouse was used as a negative control (designated Negative Control on FIG. 6).

Fusions were performed using three of the mice resulting in positive clones. Splenocytes from the mouse designated 938-3 on FIG. 6 were harvested for a fusion on Day 128. A total of 2.9×10⁸ spleen cells were mixed with 3×10⁷ NS1 myeloma cells, centrifuged to pellet the cells. The supernatant was aspirated and 1.0 ml 50% polyethylene glycol in media (PEG) was added drop wise over 1 minute to allow cell membranes to fuse. Culture media was then added drop wise to dilute the PEG 20-fold. The fused cells were centrifuged and resuspended in 100 ml Selection media consisting of Dulbecco's Modified Eagle Medium high glucose (DMEM) base with added 15% fetal bovine serum (FBS), 100 uM hypoxanthine, 2% (v/v) Hybridoma Cloning Supplement (Roche), 4 mM L-glutamine, 50 ∪/ml penicillin, 50 μg/ml streptomycin, 1× nonessential amino acids and the selective agent azaserine at 5.7 uM used to kill unfused NS1 cells. The cell suspension was dispensed into 24 well plates, 1 ml/well. Plates were incubated at 37° C. in 8% CO₂ atmosphere for 6 days and then fed with 1 ml per well of Growth media consisting of the components described above minus the azaserine. Cells were allowed to grow an additional 4-5 days.

On Day 11 post fusion, cell supernatants were tested for the presence of anti-staphylocoagulase and/or vWbp peptide antibodies, respectively, by ELISA using the ELISA assay described above. The positive well was subcloned by the limiting dilution method in Growth media to obtain a monoclonal cell line.

For this method cells were seeded into 96-well plates at sufficiently low density to increase the probability of a colony growing from a single cell. Approximately 10 days later, wells were screened by solid-phase ELISA to identify antibody-producing clones. The contents of the positive well were subcloned again by limiting dilution seeding into a single 96 well plate. The contents of the positive wells were given the designations 2A1.5H4.B7 (anti-SC GMA-2105), 10C9.D1.B6 (anti-vWbp GMA-2500), and 15H9.E1.A2 (anti-vWbp GMA-2510), respectively. The cells were grown to sufficient numbers for cryopreservation and antibody production.

For antibody production, cells were grown to high density in roller bottles in 500 ml serum-free media. Antibody was purified from the supernatant by allowing the antibody to bind to protein G Sepharose on an affinity column (Pierce) that allows non-antibody proteins to flow through. Bound antibodies were then eluted from the Sepharose using 0.1 M glycine, pH 2.7. Eluted antibodies were neutralized to pH 7 with 1 M Tris buffer. A buffer transfer into phosphate buffered saline (PBS) was performed by dialysis using 30 kd molecular weight cut off dialysis tubing. Purified antibody was stored at 4° C. in the presence of 0.1% sodium azide as a preservative.

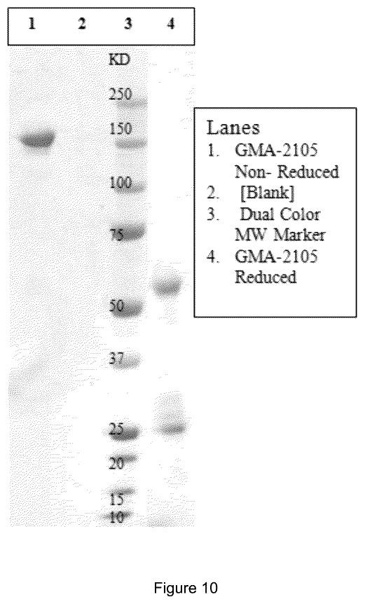

FIG. 10 is an SDS-PAGE image of purified GMA-2105. An antibody sample of 3 μg was electrophoresed at 200V on a 4-12% Bis-Tris gel under reducing and non-reducing conditions. Lane 1 shows GMA-2105 under non-reduced conditions resulting in a single band of molecular weight 155 kDa. Lane 4 shows GMA-2105 under reduced conditions with about 55 kDa (heavy chain) and 26 kDa (light chain). Lane 3 refers to the molecular weight marker. Lane 2 is empty.

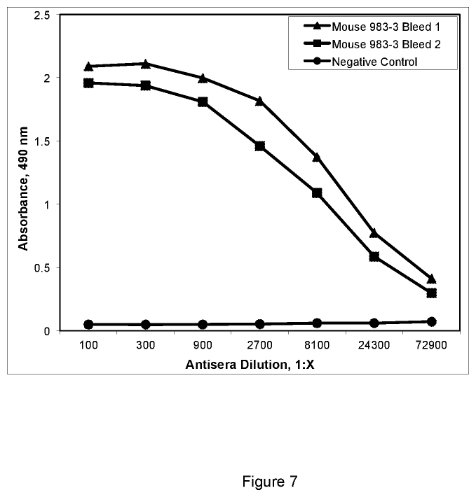

FIG. 7 shows the serum immune response from the splenocyte donor mouse that was used for hybridoma production of the anti-SC antibody. The splenocyte donor mouse was bled at two time points. Binding of mouse serum antibody to staphylocoagulase peptide SC(1-10) (SEQ ID NO.: 9) ovalbumin coated wells of a 96 well plate was detected using a goat anti-mouse secondary antibody conjugated to horseradish peroxidase. Substrate ortho-phenylenediamine conversion to product was measured at 490 nm using a spectrophotometer. The negative control was the serum from an irrelevant mouse.

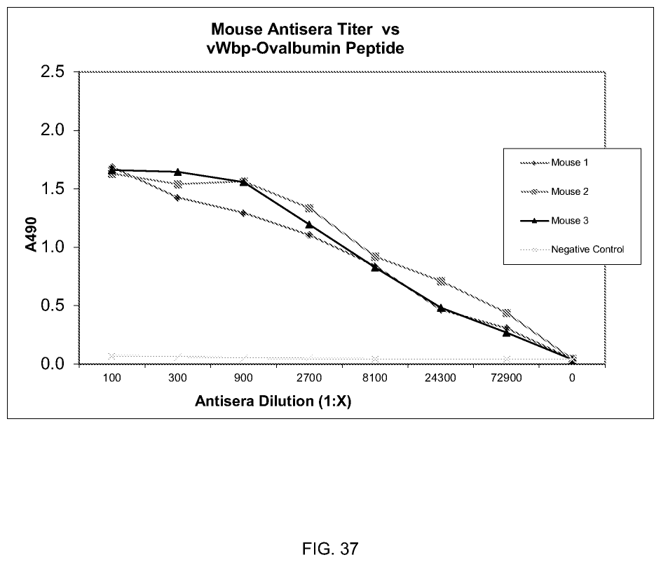

FIG. 37 shows the serum immune response from the splenocyte donor mouse that was used for hybridoma production of the anti-vWbp antibodies. The splenocyte donor mouse was bled at two time points. Binding of mouse serum antibody to vWbp peptide vWbp(1-12) (SEQ ID NO.: 28) ovalbumin coated wells of a 96 well plate was detected using a goat anti-mouse secondary antibody conjugated to horseradish peroxidase. Substrate ortho-phenylenediamine conversion to product was measured at 490 nm using a spectrophotometer. The negative control was the serum from an irrelevant mouse.

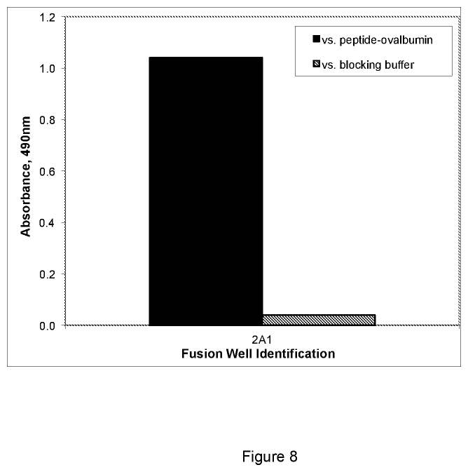

FIG. 8 shows the binding of anti-staphylocoagulase antibody GMA-2105 in hybridoma cell supernatant to SC(1-10) peptide ovalbumin conjugate bound to the wells of a 96 well plate. The antibody-antigen complex was detected using a goat anti-mouse secondary antibody conjugated to horseradish peroxidase. Substrate ortho-phenylenediamine conversion to product was measured at 490 nm using a spectrophotometer. The negative control was a well that was coated with carbonate buffer with no antigen and then blocked with blocking buffer. The negative control well did not contain the peptide conjugate.

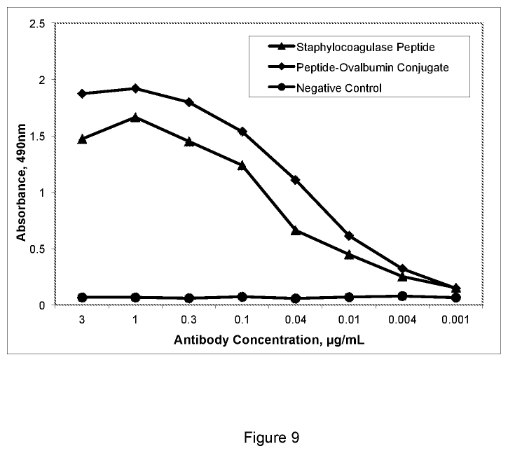

FIG. 9 demonstrates the ability of the purified monoclonal antibody GMA-2105 disclosed herein to bind to the staphylocoagulase peptide SC(1-10) (residues 1-10 of SEQ ID NO.: 2). For ease of reference, the purified antibody was designated GMA-2105. The curve shows binding of GMA-2105 to unconjugated staphylocoagulase peptide SC(1-10)(SEQ ID NO.: 9), and staphylocoagulase (SEQ ID NO.: 2) -ovalbumin conjugate. A purified, irrelevant mouse IgG was used as a negative control.

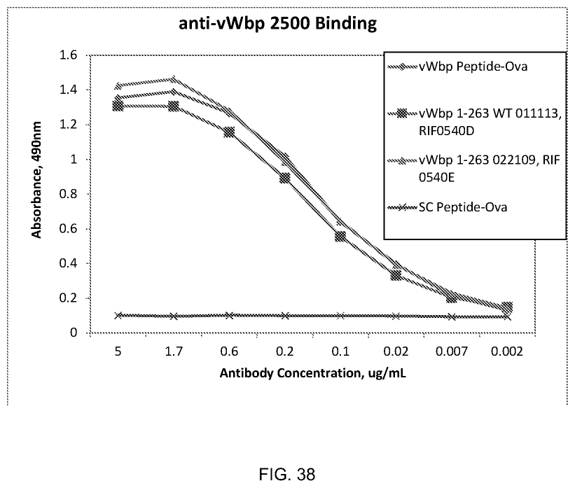

FIG. 38 demonstrates the ability of the purified monoclonal antibody GMA-2500 disclosed herein to bind to the vWbp peptide vWbp(1-12) (residues 1-12 of SEQ ID NO.: 4). For ease of reference, the purified antibody was designated GMA-2500. The curve shows binding of GMA-2500 to unconjugated staphylocoagulase peptide vWbp(1-12)(SEQ ID NO.: 28), and vWbp (SEQ ID NO.: 4) -ovalbumin conjugate. A purified, irrelevant mouse IgG was used as a negative control.

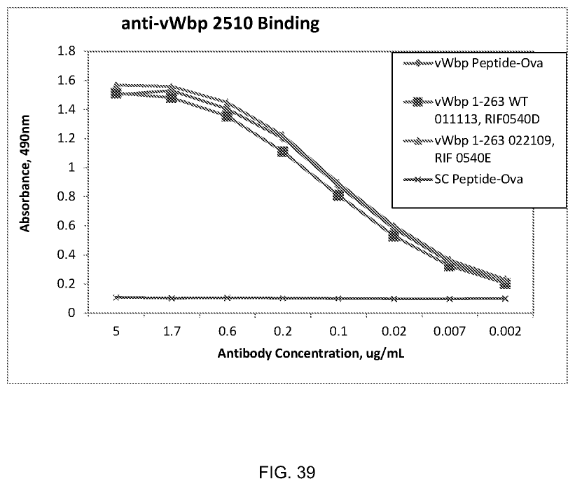

FIG. 39 demonstrates the ability of the purified monoclonal antibody GMA-2510 disclosed herein to bind to the vWbp peptide vWbp(1-12) (residues 1-12 of SEQ ID NO.: 4). For ease of reference, the purified antibody was designated GMA-2510. The curve shows binding of GMA-2510 to unconjugated staphylocoagulase peptide vWbp (1-12)(SEQ ID NO.: 28), and vWbp (SEQ ID NO.: 4) -ovalbumin conjugate. A purified, irrelevant mouse IgG was used as a negative control.

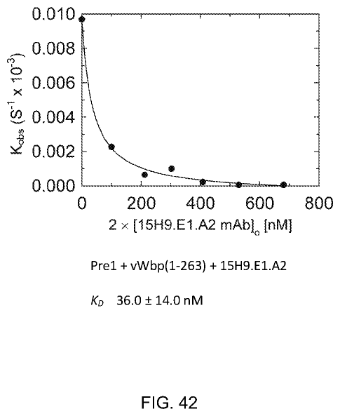

FIG. 42 demonstrates activity assays of prethrombin-1 (1 nM) with vWbp(1-263) (10 nM) in the presence of increasing concentration of 15H9.E1.A2 mAb (anti-vWbp GMA-2510) (subunit concentration) (2×[Murine 15H9.E1.A2 mAb]ₒ) in Ca²⁺ buffer, pH 7.4 (50 mM HEPES, 110 mM NaCl, 5 mM CaCl₂ and 1 mg/ml PEG-8000). The activity curves were hysteretic. The curves were analyzed using hysteretic equation to obtain K_{obs}. The K_{obs} is plotted as a function of 15H9.E1.A2 mAb (anti-vWbp GMA-2510) (subunit concentration) (2× [Murine 15H9.E1.A2 mAb]ₒ) using bindf-1k equation to get a K_{D}.

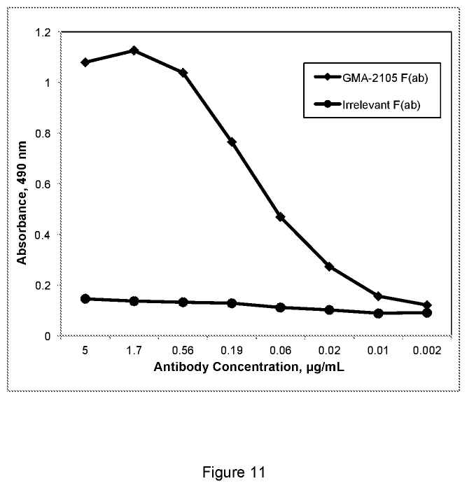

FIG. 11 demonstrates the successful binding of GMA-2105 fragment antigen-binding fragment (F(ab) fragment). The GMA-2105 F(ab) was tested by ELISA against the IVTKDYSKES peptide (SEQ ID NO.: 9). The negative control was a purified irrelevant F(ab) fragment. This demonstrates that the GMA-2105 F(ab) fragment, alone, is capable of binding staphylocoagulase IVTKDYSKES peptide (SEQ ID NO.: 9).

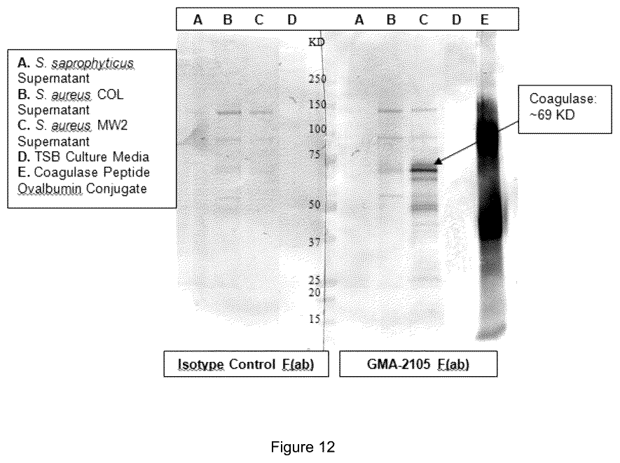

FIG. 12 shows a Western blot detection of staphylocoagulase in cultured bacteria of various strains using purified GMA-2105 F(ab) fragments. Lanes A, B, and C contain concentrated (10x) supernatants from identically cultured bacteria. Lane A: S. saprophyticus (coagulase negative bacterium control); Lane B: S. aureus strain COL (MRSA); Lane C: S. aureus strain MW2 (MRSA); Lane D: media blank; Lane E: staphylocoagulase peptide conjugated to ovalbumin (SEQ ID NO.: 9). The Western blot demonstrated the ability of the GMA-2105 F(ab) fragments to bind staphylocoagulase, even in cultured bacteria.

Experiment: Demonstration of GMA-2105 Binding to Staphylocoagulase Variants

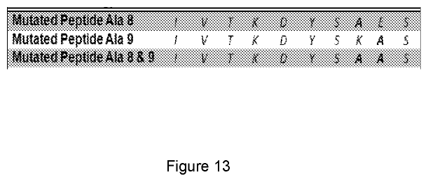

There are reported strain specific sequence differences at residue positions 8 and 9 of staphylocoagulase (SEQ ID. No.: 2). Specifically, various strains have been found to have amino acid substitutions at positions 8 and/or 9. Wantabe, et al., Genetic Diversity of Staphylocoagulase Genes (coa): Insight into the evolution of variable chromosomal virulence factors in Staphylococcus aureus. PLOS ONE 2009; 4(5): 1-11, incorporated herein by reference in its entirety. To demonstrate the ability of GMA-2105 to recognize and bind to variations of staphylocoagulase among different S. aureus strains, we synthesized three peptides with alanine substitutions at positions 8 of SEQ ID NO. 9 (“Mutated Peptide Ala 8”), position 9 of SEQ ID NO. 9 (“Mutated Peptide Ala 9”), and position 8 and 9 of SEQ ID NO.: 9 (“Mutated Peptide Ala 8 & 9”) (see FIG. 13). We measured the binding of each synthetic peptide (Mutated Peptide Ala 8, Mutated Peptide Ala 9, and Mutated Peptide Ala 8 & 9) to GMA-2105 in solution.

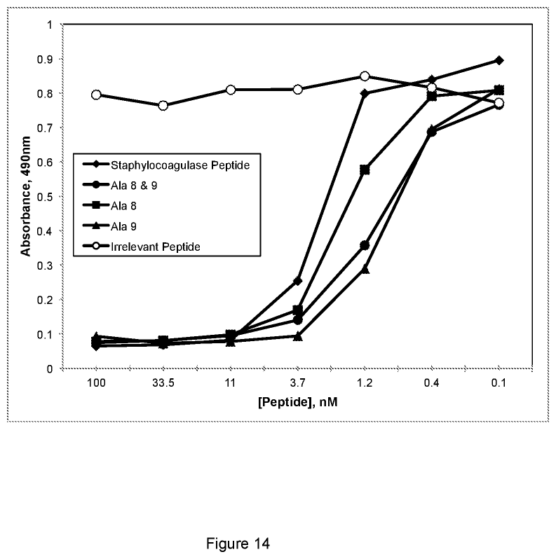

FIG. 14 shows the results of the competitive binding assay. We tested binding of Mutated Peptide Ala 8, Mutated Peptide Ala 9, and Mutated Peptide Ala 8 & 9 to GMA-2105 in a solid phase competitive inhibition assay. GMA-2105 was incubated with increasing concentrations of each of Mutated Peptide Ala 8, Mutated Peptide Ala 9, and Mutated Peptide Ala 8 & 9 (referred to hereafter as “GMA-2105 - Mutated Peptide samples”) in solution overnight at 4 degrees C. The incubation was followed by transfer of the GMA-2105 -Mutated Peptide samples to wells coated with the non-mutated peptide, IVTKDYSKES (SEQ ID. No.: 9) and blocked with 0.1% BSA PBS. After a 30-minute incubation, wells were washed with PBS containing 0.05% TWEEN 20, followed by addition of goat anti-mouse secondary antibody conjugated to horseradish peroxidase. Substrate ortho-phenylenediamine conversion to product was measured at 490 nm using a spectrophotometer. The negative control as was a purified irrelevant synthetic peptide.

Mutated Peptide Ala 8, Mutated Peptide Ala 9, and Mutated Peptide Ala 8 & 9, all bound antibody in solution. As shown in FIG. 14. Mutated Peptide Ala 8, Mutated Peptide Ala 9, and Mutated Peptide Ala 8 & 9 each and all inhibited binding of antibody onto wells coated with IVTKDYSKES (SEQ ID NO.: 9) in competitive assay.

As demonstrated in the example, we provide an isolated monoclonal antibody, and/or a purified monoclonal antibody. The antibody may be used in, for example, methods of inhibiting activation of prothrombin, for example but not limited to, inhibiting the binding of SC and/or vWbp to prothrombin. In additional to the methods described above, monoclonal antibodies may be produced using a variety of methods, e.g., methods found in Monoclonal Antibodies: Methods and Protocols, ISBN: 1588295672; Antibodies: A Laboratory Manual, ISBN: 0879693142, incorporated herein by reference in their entirety.

Resulting antibodies may be prepared in a number of forms, including chimeric, humanized, or human in addition to murine. Monoclonal antibodies may be prepared from a single chain, such as the light or heavy chains. Alternatively, or additionally, monoclonal antibodies may be prepared from active fragments, e.g., fragments that retain the binding characteristics (e.g., specificity and/or affinity) of the whole antibody. Antisera prepared using monoclonal or polyclonal antibodies provided herein may be prepared in a number of suitable ways.

### EXAMPLE Inhibition of Prothrombin Activation

A high affinity monoclonal antibody GMA-2105 targeting the staphylocoagulase N-terminus residues inhibits prothrombin activation. In solution, the antibody bound Ser 7 to Cys mutant of SC(1-325). Binding studies were done using the BODIPY-labeled staphylocoagulase SC (S7C)(1-325) (SEQ ID NO.: 2, positions 1-325 with Cys at position 7). The change of fluorescence upon antibody binding is measured as a function of the unlabeled ligand concentration and fitting the data to the quadratic binding equation gives the dissociation constant (K_{D}), and the stoichiometry (n). Panizzi, et al., Fibrinogen substrate recognition by staphylocoagulase-(pro)thrombin complexes, J. Biol. Chem. 2006; 281:118-95.

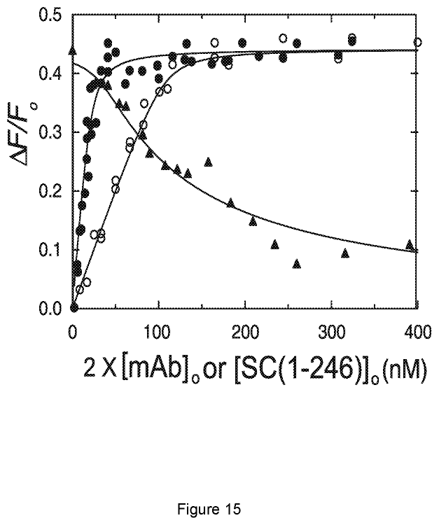

Analysis of the increase in BODIPY fluorescence on GMA-2105 binding gave K_{D}2 nM for binding of 1.6 mol BODIPY-SC/mol GMA-2105, indicating that the antibody GMA-2105 is bivalent and binds the staphylocoagulase SC(1-246)(positions 1-246 of SEQ ID NO.: 2) with high affinity. Binding of the labeled staphylocoagulase SC(1-246) (positions 1-246 of SEQ ID NO.: 2) to the GMA-2105 was competitively inhibited by binding of an unlabeled staphylocoagulase peptide representing positions 1-246 (SC(1-246)) of SEQ ID NO.: 2, with K_{D} 4 nM (descending curve in FIG. 15), indicating comparable affinity of the antibody for staphylocoagulase (positions 1-246 of SEQ ID NO.: 2).

FIG. 15 shows equilibrium binding of GMA-2105 to staphylocoagulase SC(1-246) (positions 1-246 of SEQ ID NO.: 2). The results indicate high-affinity interaction. Direct binding of BODIPY-SC(S7C) at 27nM (●) and 130 nM (∘) probe titrated with GMA-2105 caused fluorescence increase, whereas unlabeled SC(1-246) (triangles) displaced BODIPY-SC(S7C) labeled staphylocoagulase from binding the antibody. Simultaneous fit of all datasets provide the Kd for the unmodified competitor to the antibody.

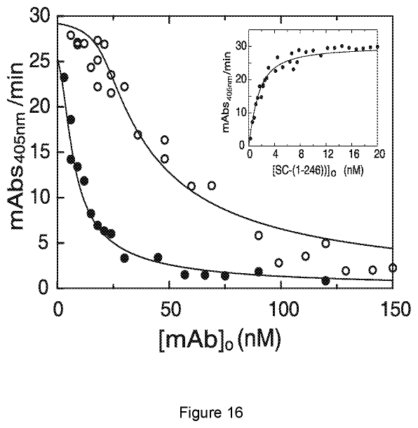

The GMA-2105 antibody inhibits prothrombin30 activation by SC(1-246) (positions 1-246 of SEQ ID No. 246) with 0.9 nM affinity (FIG. 16). Inhibition of prothrombin activation is complete as determined by analysis of inhibition at two SC(1-246) (positions 1-246 of SEQ ID No. 246) concentrations as competitive binding of prothrombin³⁰ and GMA-2015 to SC(1-246) (positions 1-246 of SEQ ID No. 246), meaning that staphylocoagulase bound to the GMA-2105 cannot activate prothrombin.

Using fluorescently labeled staphylocoagulase (SC(1-325) and Ser to Cys at position 7 of SEQ ID No. 2), and unlabeled GMA-2105, gave a K_{D} of 0.99±0.07 nM and a stoichiometry of 2 mol staphylocoagulase/mol GMA-2105. Concentrations were: BODIPY-staphylocoagulase (1-325) at 27 nM (●) and 130 nM (∘) titrated with GMA-2105 and unlabeled staphylocoagulase (1-246)(residues 1-246 of SEQ ID No. 2).

FIG. 16 shows inhibition of SC(1-246)●prothrombin^{3Q} by GMA-2105. This data gave identical K_{D} and n for antibody binding to staphylocoagulase. Rates of S2238 hydrolysis by prothrombin (1nm) and SC(1-246) at 5.6nM(●) and 28 nM (∘) versus GMA-2105 concentration. The inset shows titration of 1nM prothrombin with SC(1-246).

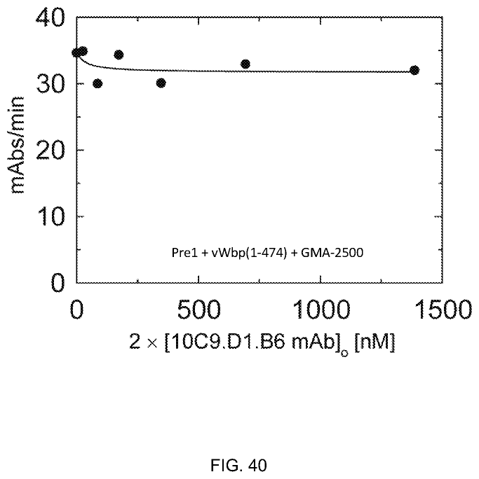

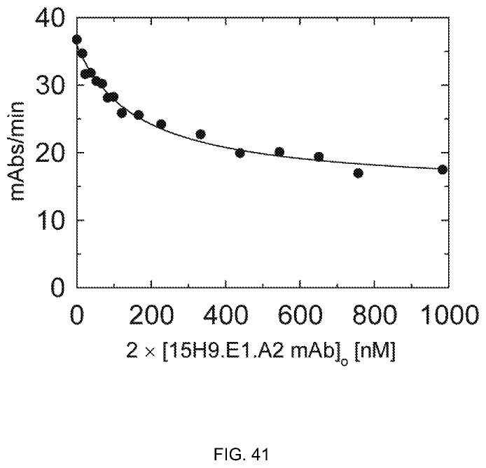

FIG. 40 provides data for GMA-2500 and FIG. 41 provides data for GMA-2510 with respect to vWbp.

FIG. 40 provides the results of activity assays of prethrombin-1 (1 nM) with vWbp(1-474) (10 nM) in the presence of increasing concentration of 10C9.D1.B6 mAb (anti-vWbp GMA-2500) (subunit concentration) (2× [Murine 10C9.D1.B6 mAb]ₒ) in Ca²⁺ buffer, pH 7.4 (50 mM HEPES, 110 mM NaCl, 5 mM CaCl₂ and 1 mg/ml PEG-8000). The activity curves were linear. The linear rates are plotted as a function of 10C9.D1.B6 mAb (anti-vWbp GMA-2500) (subunit concentration) (2× [Murine 10C9.D1.B6 mAb]∘) using bindf-1k equation.

FIG. 41 provides the results of activity assays of prethrombin-1 (1 nM) with vWbp(1-474) (10 nM) in the presence of increasing concentration of anti-vWbp 15H9.E1.A2 (GMA-2510) anti-vWbp mAb (subunit concentration) (2× [Murine GMA-2510 mAb]∘) in Ca²⁺ buffer, pH 7.4 (50 mM HEPES, 110 mM NaCl, 5 mM CaCl₂ and 1 mg/ml PEG-8000). The activity curves were linear. The linear rates are plotted as a function of 15H9.E1.A2 (GMA-2510) anti-vWbp mAb (subunit concentration) (2× [Murine 15H9.E1.A2 mAb]∘) using bindf-1k equation to get a K_{D}

EXAMPLE: Inhibition of plasma clotting.

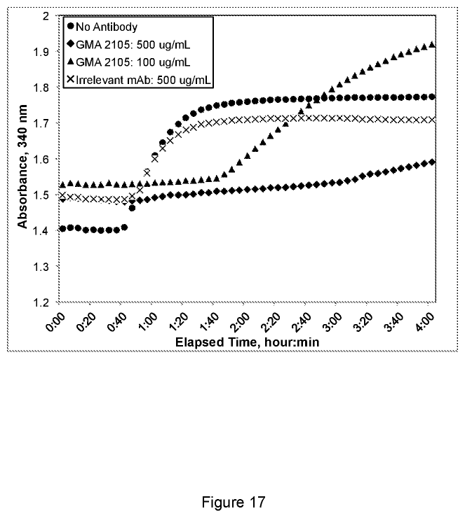

GMA-2105 inhibits S. aureus plasma clotting. FIG. 17 shows the inhibition of plasma clotting by increasing concentration of purified GMA-2105. GMA-2105 at varying concentrations was incubated with supernatant from S. aureus strain Tager 104 grown to OD 600 of 0.5 for one hour at room temperature followed by addition of an equal volume of rabbit plasma. Clotting was measured using a microplate spectrophotometer at 340 nm. Addition of GMA-2105 at 100 ug/mL (triangles) and 500 ug/mL (diamonds) prevented clotting as shown. The controls included an isotype matched antibody (XX) and no antibody (solid circle).

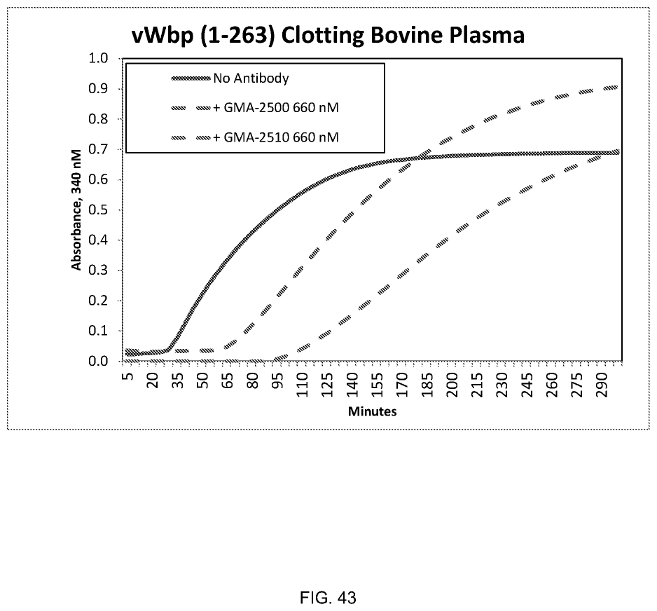

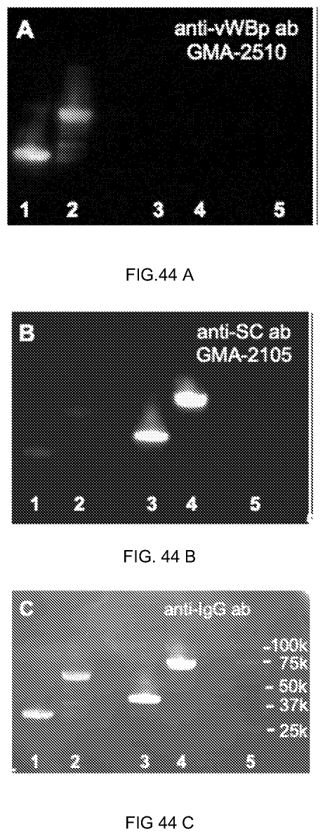

The anti-vWbp antibodies designated GMA-2500 and GMA-2510 inhibit S. aureus plasma clotting. FIG. 43 shows the inhibition of plasma clotting by purified GMA-2500 and GMA-2510.

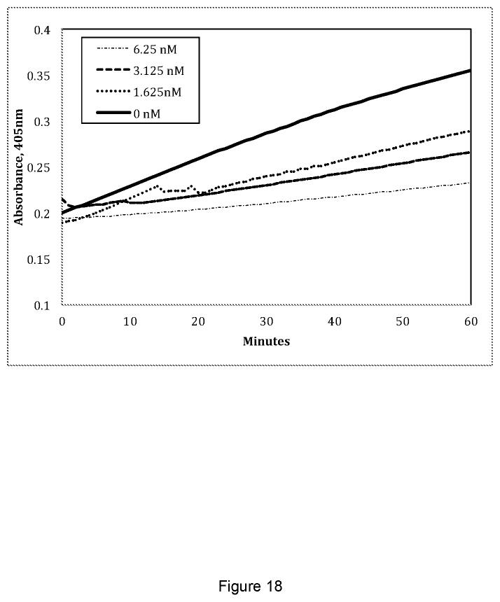

FIG. 18 demonstrates the effect of increasing concentrations of GMA-2105 to inhibit staphylocoagulase activity in S. aureus Tager supernatant. S. aureus Tager supernatant was incubated with or without GMA-2105 antibody at various concentrations (6.25 nM GMA-2105, 3.125 nM GMA-2105, 1.625 nM GMA-2105, OnM GMA-2105) for three hours at room temperature in a buffer of 50 nM HEPES pH 7.4, 150 nM NaCl, 5 mM CaCl₂, 1mg/mL PEG8000. Prothrombin was added to a 40 nM final concentration. The samples were incubated for three hours at room temperature. After incubation, S-2238 chromogenic substrate was added to a final concentration of 286 uM and the resulting absorbance was read at 405 nM for 1 hour.

EXAMPLE: GMA-2105 Antibody does not aggregate.

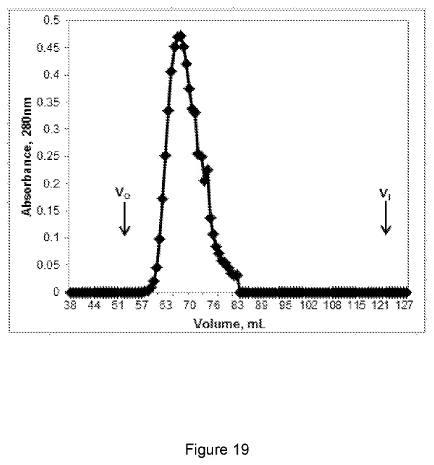

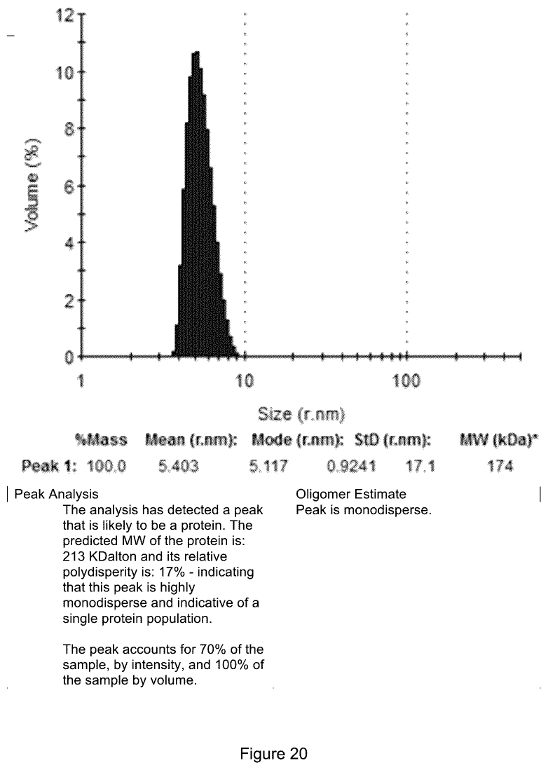

Protein therapeutics, including monoclonal antibodies, have the potential for instability in which the physical state of the protein changes even through the chemical composition is unaltered. Several clinical trials with monoclonal antibodies have failed due to antibody aggregation. Two methods are commonly used to measure protein aggregation: size exclusion chromatography and light scattering. FIGS. 19 and 20 demonstrate evidence using these methods that isolated GMA-2105 does not aggregate.

FIG. 19. Purified GMA-2105 was analyzed by size exclusion chromatography using a column packed with SEPHACRYL 300 resin. A total of 4.2 mgs or 5.9 absorbance units were loaded onto the column in a volume of 1.5 mL. Fractions (1 mL) were collected and absorbance determined by spectrophotometry at 280 nM. Recovery off the column was 5.6 absorbance units, accounting for 96 percent of the sample that was loaded.

FIG. 20. Dynamic light scattering using MALVERN ZETASIZER NANO S at 25.0 degrees Celsius. A 1 mL fraction from the size exclusion chromatography analysis was filtered using a WHATMAN 0.2 uM filter, loaded into a disposable sizing cuvette and run using protein analysis software. GMA-2105 appears to be adequately stabilized in solution following production and purification.

EXAMPLE: Survival Study and Pharmacokinetics

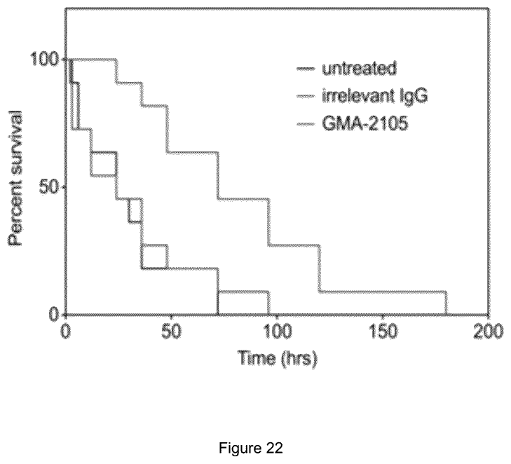

The mAb targeting SC(1-10) characterized in Preliminary Results promotes survival in a mouse model of S. aureus sepsis. Injection of purified antibody into mice followed by challenge with S. aureus gave the survival curve shown in FIG. 22. The experiment shows statistically significant survival over the controls (irrelevant antibody or phosphate-buffered saline).

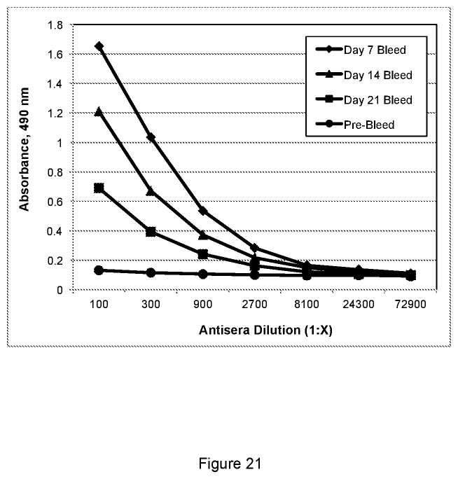

FIG. 21. Detection of GMA-2105 antibody in the blood of mice 21-day post administration. C57BL/6 mice were injected intraperitoneal with 120 ug of the mAb against SC(1-10) (GMA-2105). Every 7 days for a total of 21 days, blood was collected from the tail vein of each of the three mice. The antisera was diluted and added to a 96 well ELISA plate coated with the peptide IVTKDYSKES (SEQ ID. No.: 9) and blocked with 0.1% BSA/PBS. After a 1-hour incubation, wells were washed with PBS containing 0.05% TWEEN 20 followed by addition of goat anti-mouse secondary antibody conjugated to horseradish peroxidase. Substrate orth-phenylenediamine conversion to product was measured at 490 nm using a spectrophotometer.

FIG. 22. Survival Curve. C57BL/6 mice were injected intraperitoneal with 120 ug of the mAb against SC(1-10) (GMA-2105) or an isotype-matched irrelevant mAb or PBS buffer with no antibody. After 8-9 hours, treated and untreated mice were injected with 1×108 CFU of S. aureus via tail vein and monitored for survival. The anti-SC(1-10) mAb GMA-2105 increased the median survival curves from 24 to 72 h (p<0.005 vs. untreated).

Chimeric GMA-2105

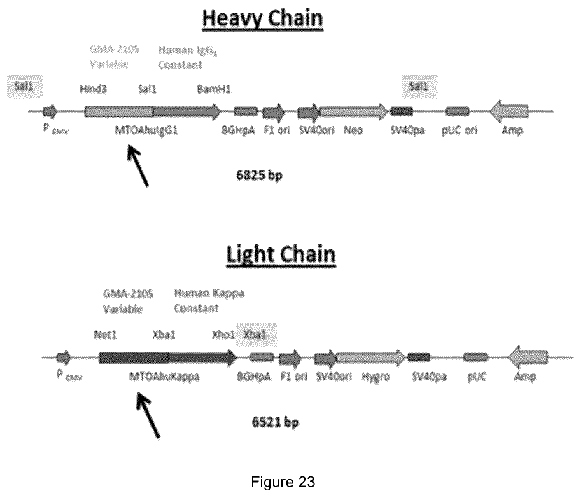

Chimeric GMA-2105 was made by sequencing cDNA from purified mRNA from GMA-2105 hybridoma cells and using this cDNA sequence to synthesize DNA fragments with appropriate restriction sites at the ends. The DNA segments were inserted into expression vectors containing the human constant regions. FIG. 23 provides a schematic. SEQ ID No. 20 provides an exemplary human IgG₁ CH1, SEQ ID No. 21 provides an exemplary human IgG₁ hinge region; SEQ ID No. 22 provides an exemplary human IgG₁ CH2; SEQ ID No. 23 provides an exemplary human IgG₁ CH3, SEQ ID No. 24 provides an exemplary human IgG₁ kappa light chain constant region.

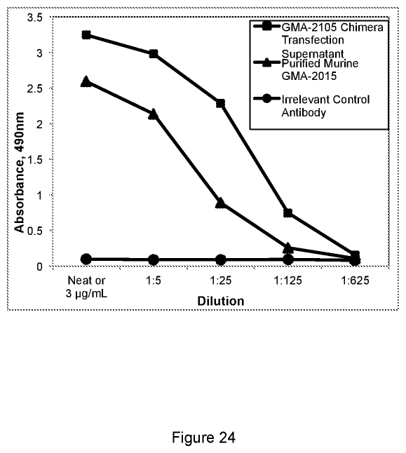

The ability of the GMA-2105 antibody chimera to bind staphylocoagulase was tested by analyzing the supernatants of the transfected cells. FIG. 24 provides the results of binding of GMA-2105 chimera in HEK293 cell transfection supernatant to IVTKDYSKES (SEQ ID No. 9) in an ELISA assay. A combination of 55 μg of GMA-2105 heavy chain chimera DNA and 57 μg of GMA-2105 light chain chimera DNA was mixed with 293fectin and added into 6×10⁷ HEK293 cells in a total volume of 60 mL following Invitrogen's Freestyle 293 Expression system transfection protocol. The cells were incubated at 37 degrees C. with shaking to allow for antibody production and the supernatant was harvested 5 days post transfection. Antibody-antigen complex was detected using a goat anti-human secondary antibody conjugated to horseradish peroxidase for transfection supernatant or goat anti-mouse conjugated to horseradish peroxidase for purified murine GMA-2105 antibody detection. Substrate ortho-phenylenediamine conversion to product was measured at 490 nm using a spectrophotometer. Negative control was purified, irrelevant human IgG.

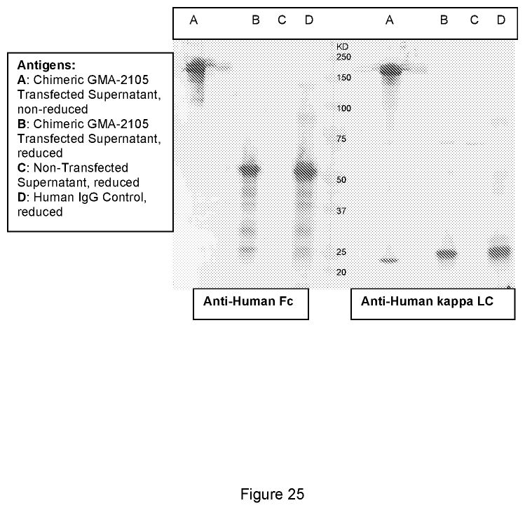

The expression of recombinant chimeric GMA-2105 antibodies in supernatants were analyzed with SDS-PAGE, shown as FIG. 25. Detection of human antibody heavy chain Fc and human kappa light chain in HEK293 GMA-2105 chimeric HC and LC transfected supernatant. Supernatants were electrophoresed on NOVEX NUPAGE 4-12% Bis-Tris gel at 200 V followed by electrophoretic transfer to nitrocellulose and blocking with 1% BSA in PBS. Antibody in supernatant was detected using a biotinylated anti-human kappa chain antibody (Vector Laboratories) or biotinylated anti-human IgG, gamma chain specific antibody (Vector Laboratories) followed by avidin peroxidase with chromogenic substrate. Negative control was non-transfected HEK cell supernatant and positive control was human IgG, 0.5 ug load. Lane A: Chimeric GMA-2105 transfected supernatant non-reduced; Lane B: Chimeric GMA-2105 transfected supernatant non-reduced; Lane C: Non-transfected supernatant reduced; Lane D: Human IgG control reduced

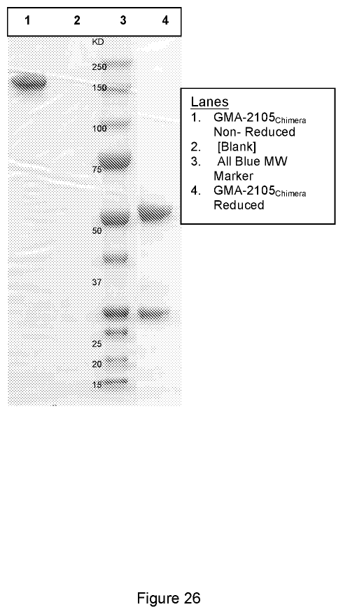

The purified chimeric GMA-2105 antibodies were analyzed with SDS-PAGE, shown as FIG. 26. An antibody sample of 3 μg was electrophoresed at 200V on a NOVEX NUPAGE 4-12% Bis-Tris gel under reducing and non-reducing conditions. After 1 hour, the gel was removed and incubated in fixative (50% ethanol.water, 7% (v/v) acetic acid) for 1 hour followed by a 2× wash with ddH₂O and inubation overnight in Gel Code Blue (Invitrogen) followed by destaining in ddH₂O.

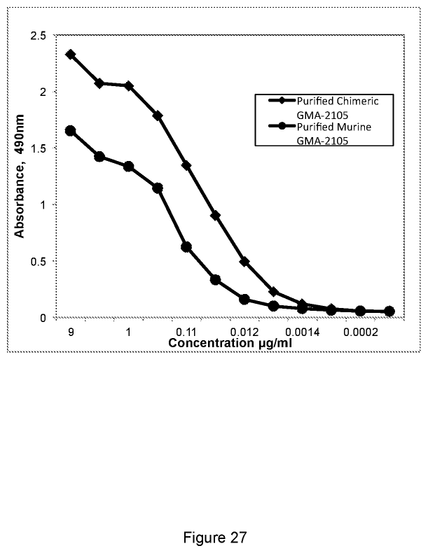

FIG. 27 shows binding of the purified chimeric GMA-2105 against staphylocoagulase. Purified chimeric GMA-2105 and murine GMA-2105 antibody were tested against staphylocoagulase peptide IVTKDYSKES (SEQ ID No.9) in an ELISA assay. The peptide IVTKDYSKES (SEQ ID No. 9) was coated onto wells of 96 well plates at a concentration of 0.5 μg/mL un-conjugated. The plate was washed then blocked with 0.1% BSA followed by addition of antibody dilutions. Antibody-antigen complex was detected using a goat anti-mouse secondary antibody conjugated to horseradish peroxidase or goat anti-human secondary antibody conjugated to horseradish peroxidase. Substrate ortho-phenylene diamine conversion to product was measured at 490 nm using a spectrophotometer

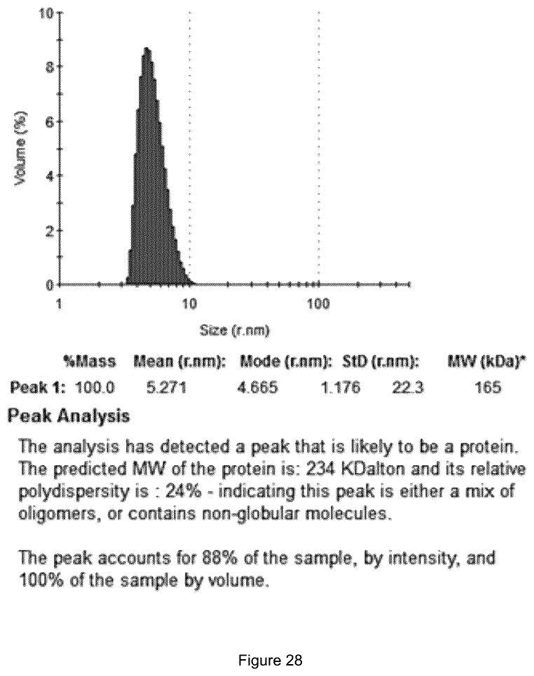

The chimeric GMA-2105 antibody was purified by affinity chromatography and analyzed by dynamic light scattering to rule out aggregation. In FIG. 28 dynamic light scattering of GMA-2105 chimera using MALVERN ZETASIZER nano S at 25.0° C. The chimeric antibody sample was filtered using a WHATMAN 0.2 um filter then ˜100 ul was loaded into a disposable low-volume sizing cuvette and run using software version 6.20 protein analysis mode.

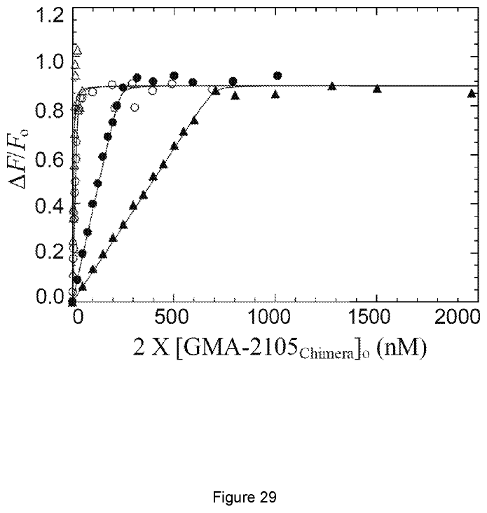

In FIG. 29, BODIPY®-labeled staphylocoagulase fragment (1-325) at 27 nM (●) and 130 nM (○) titrated with chimeric GMA-2105 in solution in the presence of unlabeled staphylocoagulase fragment (1-246) (▴).

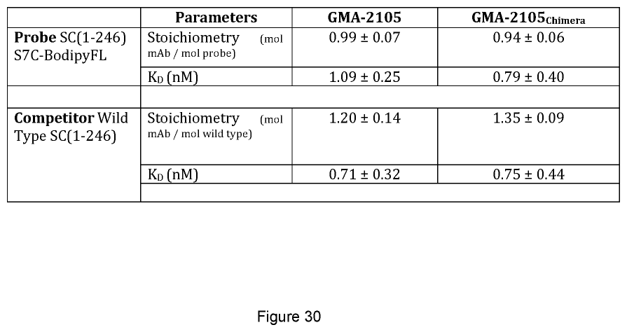

FIG. 30 summarizes the binding characteristics of the murine GMA-2105 and the chimeric GMA-2105. The chimeric antibody bound staphylocoagulase with a Kp of 0.79+0.40 nM and a stoichiometry of 2 mol staphylocoagulase/mol chimeric antibody. The binding characteristics of the chimeric GMA-2105 were essentially identical to values for murine GMA-2105.

Modeling and Construction of Human GMA-2105 Antibody

Murine antibody sequences were “humanized” to eliminate the human anti-mouse immune response. The first step was formation of a chimeric antibody by grafting the mouse heavy and light chain variable regions onto a human Fc region (described above). A second step was the refinement of the mouse variable regions by substituting specific amino acid residues while maintaining antigen binding of the complementarity determining regions (CDRs) and the supporting scaffold sequences. Human amino acid sequences are either consensus sequences for IgG subgroups, germline sequences, mature human antibody sequences, or sequences with a corresponding x-ray structure (See, e.g., Almagro, et al., Antibody Engineering: Humanization, Affinity Maturation, and Selection Techniques. An Z. editor. Therapeutic Monoclonal Antibodies. Hoboken, NJ: John Wiley & Son; 2009, incorporated herein by reference).

With the assistance of the Rosetta Design Group, we undertook a structure- and sequence- guided approach to humanization of the GMA-2105 variable region. BLAST analysis of the Protein Data Bank (PDB) for human homologs of GMA-2105 identified a human germline antibody (B3/3-23) as a suitable scaffold, with an available crystal structure (PDB ID:3QOS), as a recipient for the GMA-2105 CDRs (Seq ID Nos. 10-15). A small ensemble of models for the GMA-2105 variable region resulted from 3000 folding simulations. The ensemble was analyzed to determine GMA-2105 scaffold amino acids that might effect CDR conformation and hence antigen binding. After grafting the GMA-2105 CDRs (SEQ ID Nos.: 10-15) into B3/3-23, further simulations were conducted to assess the effect of back-mutations.

FIG. 31 provides a theoretical model of GMA-2105 light chain SEQ ID No.: 8, residues 4-105) and GMA-2105 heavy chain (SEQ ID No. 6, residues 4-110) using RosettaAntibody software. Model shows TT-stacking interacions among the complementarity determining regions and aromatic residues that are well packed.

FIG. 32 provides representatives from each of the 6 clusters formed from the 26 lowest energy GMA-2105 models. A notable feature in the dominant cluster is TT-stacking interacions among residues W33, R52, Y58, and Y97 of the GMA-2105 heavy chain (SEQ ID No. 6).

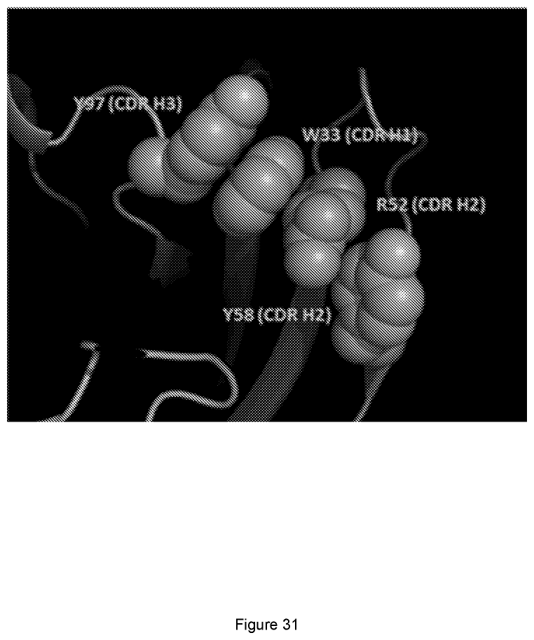

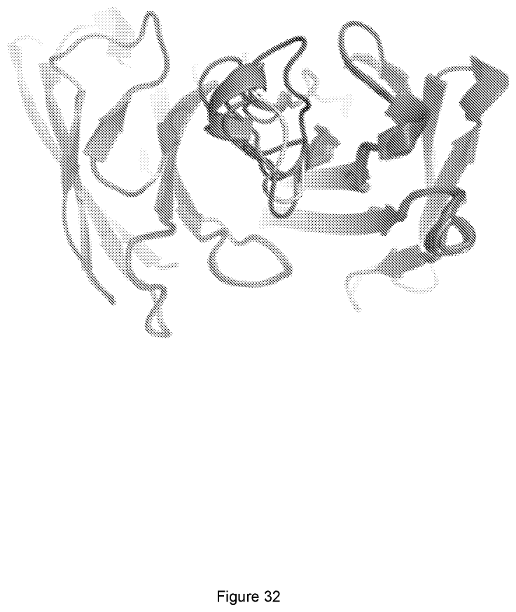

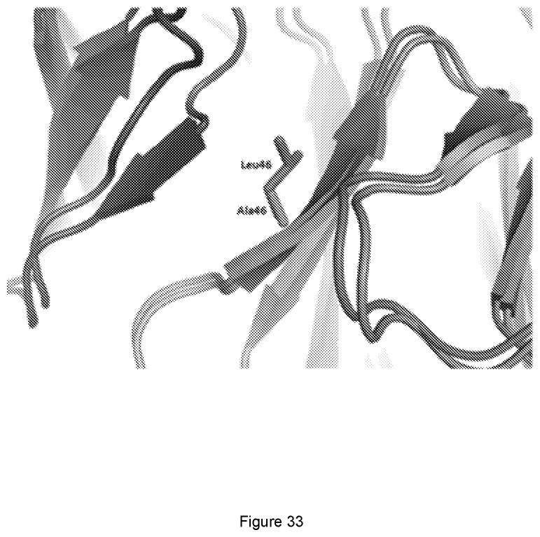

FIG. 33 shows that substitution at GMA-2105 light chain residue 46 (SEQ ID No. 8, residue 46) from alanine to leucine in the human framework may effect inter-chain orientation as this residue lies at the light-heavy chain interface.

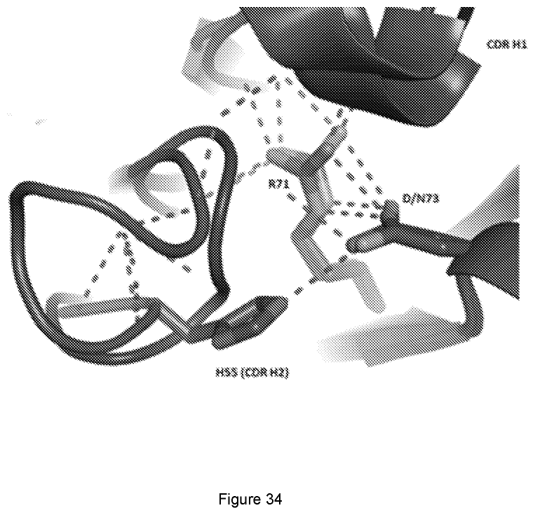

FIG. 34 shows that substituting an asparagine for aspartic acid at heavy chain residue 76 (SEQ ID NO.: 6, residue 76) may affect the folding of the heavy chain. D76 (SEQ ID NO.: 6, residue 76) may form a salt bridge with residue H58 of heavy chain CDR 2 (SEQ ID NO.: 6, residue 58 and SEQ ID. No.: 14, residue 8) and residue R74 (SEQ ID NO.: 6, residue 74) of the framework. The subsittution of asparagine at position 76 (SEQ ID NO.: 6, residue 76) forms a hydrogen bond to CDR 2 (SEQ ID NO.: 10) of the heavy chain in the human model, but it is unclear what effect other differences in the sequence would make on this interaction so the humanization GMA-2105 sequence may need to retain an aspartic acid at position 76 (SEQ ID NO.: 6, residue 76).

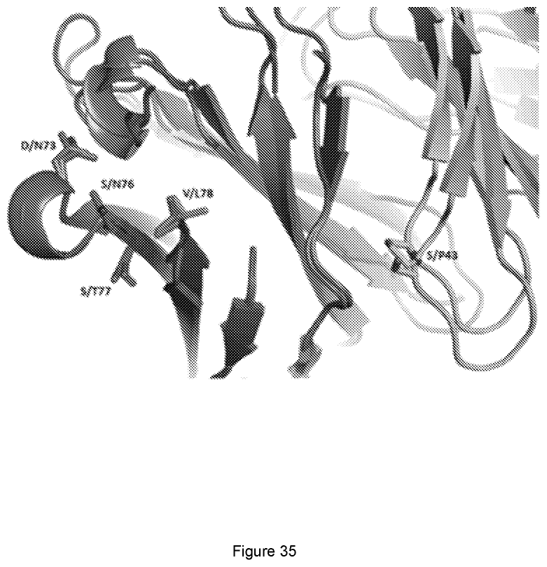

FIG. 35, subsitution of a leucine for a valine at heavy chain residue 81 (SEQ ID No. 6, residue 81) may impact the the van der Waals contact between heavy chain residue 81 and heavy chain CDR 1 residue F 29 (SEQ ID No. 6, residue 29 and SEQ ID No. 13, residue 4). Substitutions of (N for S) at heavy chain position 79 (SEQ ID No. 6, residue 79), substitution (T for S) at heavy chain position 80 (SEQ ID. No. 6, residue 80) and substitution (P for S) at light chain position 43 (SEQ ID. No. 8, residue 43) may also disrupt the contact beween heavy chain residue F29 and the H55-D73 salt bridge, so the original mouse residue may need to be retained in the humanization version.

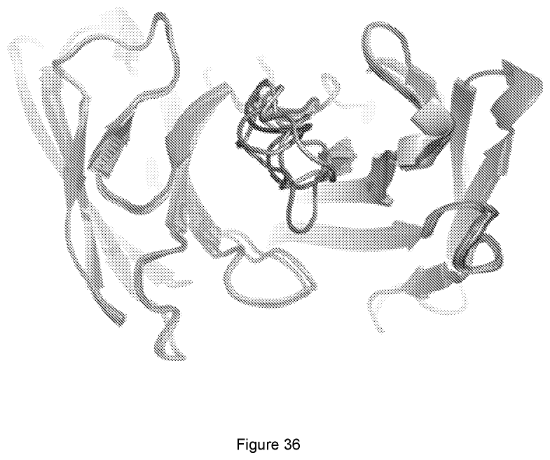

FIG. 36, the low energy models of the humanized GMA-2105 sequence. Twenty three models grouped into five clusters based on the conformation of heavy chain CDR3 (SEQ ID NO.: 15). The largest of the five clusters (16/23) clustered with the largest murine GMA-2105 cluster.

Example of Therapeutic Use of Antibodies Neutralizing SC and vWbp in mice with S. aureus endocarditis using optical imaging.

The following examples demonstrate the effectiveness of the anti-SC and anti-vWbp antibodies to deactivate S. aureus' bacterial defenses against innate immune cells. The following example also demonstrates use of anti-SC and anti-vWbp improves survival in an S. aureus animal model including a mouse model and a piglet model. This data supports the use of the disclosed antibodies as adjuvant immunotherapy for S. aureus related pathologies, including but not limited to, endocarditis. It also demonstrates the usefulness of the disclosed antibody (in conjunction with various imaging agents) to monitor S. aureus related pathologies, including but not limited to endocarditis, in large animals including humans.

Two piglet models of acute S. aureus endocarditis were employed to analyze host defense processes in large animals or humans; one affecting the tricuspid and the other the aortic valve. To mimic the typical pathogenesis of right heart endocarditis in patients, a vascular port was subcutaneously implanted into newly weaned piglets, inserting the central line into the right external jugular vein. The central line was then advanced via the superior vena cava into the right ventricle under fluoroscopy guidance. Six hours after implantation, 4-8×10⁸ CFU bioluminescent S. aureus was injected into the port. Over the course of 10 days, piglets developed typical clinical signs of endocarditis, including fever (103.5-106.5° F.) and heart murmurs. Bacteria presence on the porcine tricuspid valve was verified by hematoxylin and eosin staining and staphylococcal cells were identified by Gram staining.

The S. aureus endocarditis piglet model was subsequently characterized by clinical cardiac MRI on a 3 Tesla human scanner. The development of two to four differently sized tricuspid endocarditis lesions in the right ventricles of piglets was observed on days 10-11 after bacterial injection. Autopsy confirmed endocarditis vegetations, which were morphologically reminiscent of those typically found in patients. Further, administering fluorescent DAB-VT680XL into the piglet's ear vein allowed visualizing endocarditis vegetations, as the probe's fluorescent signal co-localized with bacterial bioluminescence.

For induction of left-sided endocarditis, the aortic valve was damaged with a cytology brush after gaining vascular access via the carotid artery, followed by intravenous injection of 5-8×10⁸ CFU bioluminescent S. aureus . Piglets with left-sided endocarditis deteriorated clinically faster than piglets with right-sided endocarditis. These were therefore imaged on day 7 after disease induction. Cardiac MRI revealed development of aortic valve lesions. Volumetric assessment of piglet hearts with right- and left-sided endocarditis indicated that the right ventricular ejection fraction was lower in piglets with tricuspid disease. Ex vivo bioluminescence signal co-localized with lesions on autopsy, which enriched DAB-VT680XL after intravenous injection.

Antibody Immunotherapy

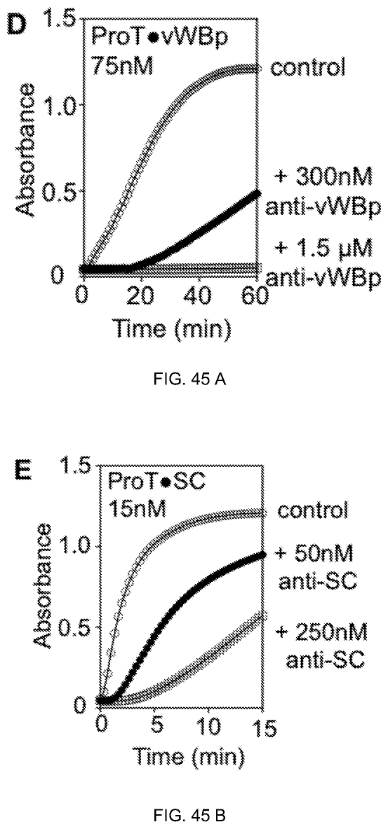

Combined antibody-mediated inhibition of SC and vWbp factors was tested and shown to disrupt S. aureus' ability to form fortified vegetations. Monoclonal antibodies raised against NH₂-terminal peptides of SC (GMA-2105) and vWbp (GMA-2510). The western blot data indicate excellent specificity and no cross-reactivity of the GMA-2105 antibody with vWbp or cross-reactivity of GMA-2510 with SC (FIG. 45, A to C). The lanes corresponded to (1) vWbp-(1-263), (2) vWbp-(1-474), (3) SC-(1-325),(4) SC-(1-660) and (5) protein standards with indicated molecular weights.

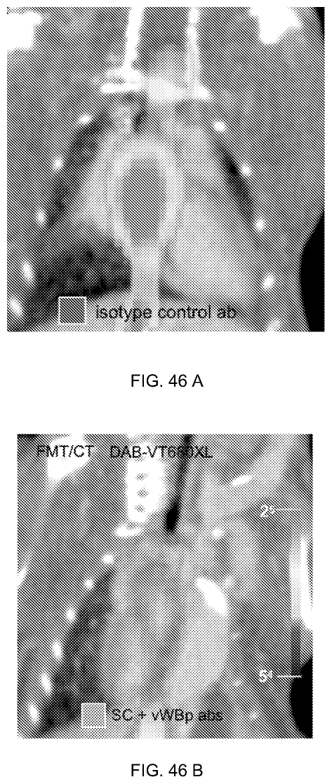

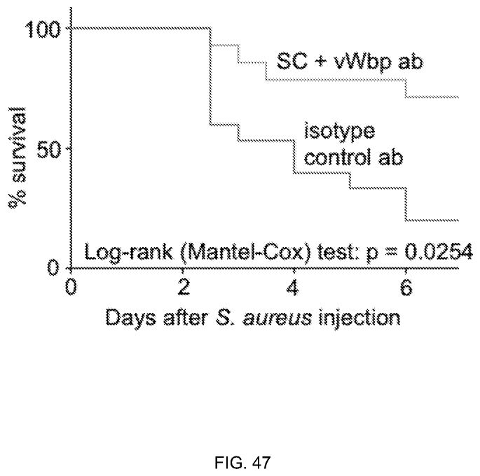

Clotting assays demonstrated that targeted antibodies reduce fibrinogen conversion to fibrin in a concentration-dependent manner for both anti-SC and anti-vWbp therapies (FIG. 45, A (GMA-2510) and FIG. 45 B (GMA-2105). Treating mice with these monoclonal antibodies led to reduced DAB-VT680XL signal in FMT/CT imaging (FIG. 46, A and B), thereby indicating that therapeutically inhibiting SC and vWbp reduces active thrombin in bacterial colonies. Injecting neutralizing antibodies improved the survival of mice with endocarditis (FIG. 47).

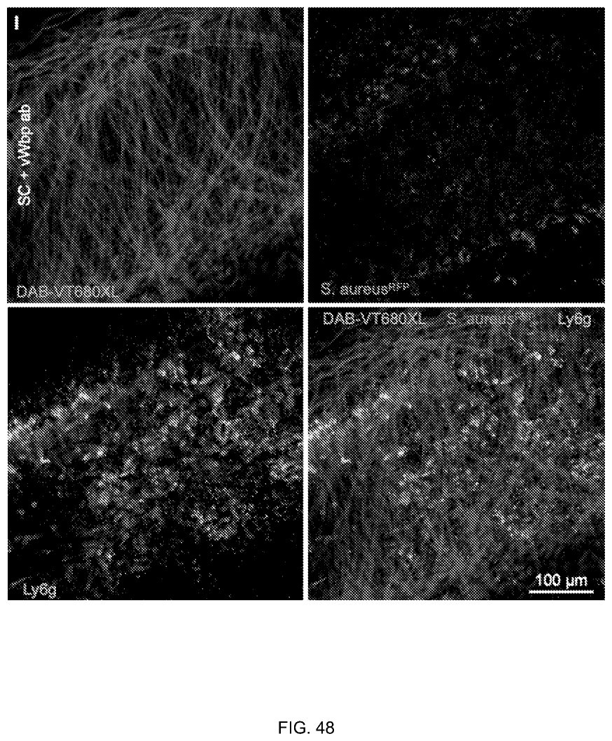

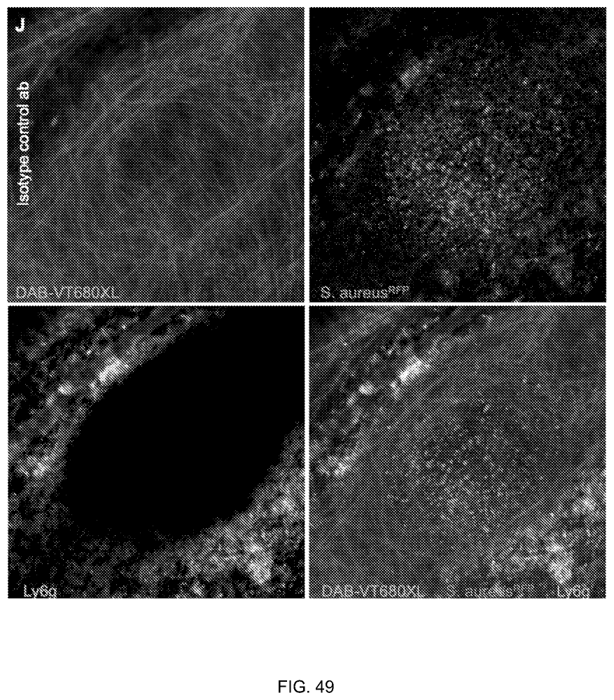

To explore how this treatment acts mechanistically, vegetations were imaged using DAB-VT680XL and intravital microscopy. The heart valves in living mice are difficult to approach with a microscope objective. S. aureus vegetations were therefore established in the femoral artery by inserting a suture and then intravenously injecting S. aureus expressing red fluorescent protein (RFP). This formed femoral artery vegetations that were comparable to the anatomy observed in the aortic valves of mice (FIG. 48 and FIG. 49). Specifically, we observed a central RFP+bacterial colony surrounded by DAB-VT680XL signal highlighting a thrombin-rich layer (FIG. 49). Staining neutrophils with intravenously injected fluorescent antibody targeting Ly6g indicated that neutrophils were unable to penetrate the capsule around RFP⁺ bacteria. Treatment with antibodies neutralizing SC and vWbp disrupted the DAB-VT680XL-stained capsule around RFP⁺ bacteria, thereby granting access to neutrophils that could then invade the S. aureus colony (FIG. 48).

In contrast to other endocarditis imaging agents, ¹⁸F-DAB and DAB-VT680XL do not directly bind bacteria but rather report on S. aureus' interaction with the host's clotting system, which contributes to biofilm formation. This fibrin-rich wall protects the bacterial colony against host immunity and—in combination with exopolysaccharides, extracellular DNA and other factors—hinders penetration of antibiotics. Data indicate that 18F-DAB and DAB-VT680XL avidly bind to this critical vegetation component, and using DAB-VT680XL in intravital microscopy visualized innate immune cells' inability to penetrate the wall, swarm into the vegetation and kill S. aureus. Interestingly, treatment with antibodies against SC (GMA-2105) and vWbp (GMA-2510) decreased DAB-VT680XL uptake, and myeloid cells were able to enter the vegetations. The microscopy data provide in vivo evidence of such host-pathogen interactions.

Three-week-old piglets were chosen because these smaller animals are easier to handle and less costly than adults. In general, swine disease models are considered similar to human pathologies and thus an attractive intermediate step for drug discovery. Because it relies on two archetypical endocarditis triggers in humans, namely a combination of an indwelling intravenous catheter or aortic valve damage with bacteremia, this animal model is particularly relevant to human disease. In addition to helping test new therapeutics and imaging approaches in a human-like setting, the piglet model could be useful for research on surgical management, a potentially life-saving treatment still in need of optimization and standardization (See, Tong, S.Y., et al., Staphylococcus aureus infections: epidemiology, pathophysiology, clinical manifestations, and management. Clin Microbiol Rev. 28(3), 603-61 (2015) incorporated herein by reference). As pig hearts are similar to humans' in size and anatomy, experiments in pigs with endocarditis could address questions such as when and how to best replace an infected valve or remove a vegetation and explore minimally invasive, catheter-based strategies.

Decreased thrombin in mouse endocarditis vegetations was detected after treatment with antibodies that neutralize SC and vWbp. In line with studies using knock-out bacteria (Mancini, S., et al., Marginal role of von Willebrand factor-binding protein and coagulase in the initiation of endocarditis in rats with catheter-induced aortic vegetations. Virulence. 9(1), 1615-1624 (2018)), it was observed that vegetations still evolved and mice succumbed eventually; nevertheless, antibody treatment prolonged survival while DAB-VT680XL signal decreased in the lesions and innate immune cells invaded bacterial colonies.

Materials and Methods

Study design. Initial optical and nuclear imaging in mice was followed by validation in a cohort of piglets subjected to either right- or left-sided endocarditis. While formal power estimations were not done, the number of mice included per study (n=6) was based on prior endocarditis imaging in mice (Panizzi, P., et al., In vivo detection of Staphylococcus aureus endocarditis by targeting pathogen-specific prothrombin activation. Nat Med. 17(9), 1142-6 (2011)). Mice were randomly assigned to disease and treatment groups. Investigators were not blinded to group allocation. Wherever possible, studies were at least done twice. The number of animals used and how many experimental replicates were performed are stated for each experiment in the figure descriptions.

Bacteria

Xen29 and Xen36 are both bioluminescent strains of coagulase-positive methicillin-susceptible Staphylococcus aureus (MSSA). Xen 43 is S. epidermidis strain. All Xen strains were purchased from PerkinElmer Inc. (Waltham, MA, USA). S. aureus^{RFP+} is a fluorescence version of methicillin-resistant S. aureus USA 300, NE1260R JE2 pckA::rfp that was obtained from Dr. Jeffery Bose of the University of Kansas Medical Center (Bose, J.L., P.D. Fey, and K.W. Bayles, Genetic tools to enhance the study of gene function and regulation in Staphylococcus aureus. Appl Environ Microbiol. 79(7), 2218-24 (2013)). Briefly, strains were cultured in liquid brain heart infusion broth under constant shaking at 150-200 rpm at 37° C. For injecting the animals, overnight cultures were diluted 20-fold in sterile Dulbecco's phosphate buffer saline without calcium or magnesium (PBS, Lonza). The injections' approximate CFU counts were assessed by light scattering at 600 nm using a Shimadzu UV-2101PC spectrophotometer according to the manufacturer's guidelines. Prior to injection, bioluminescence production was confirmed using a bioluminescence imager (FujiFilm LAS-1000) set to 10 min integration time. Post injection, true CFU numbers were verified by serial plating on 5% sheep blood agar (Hardy Diagnostics, Santa Maria, CA, USA) and expression of either the bioluminescent or fluorescent reporter gene was confirmed by imaging the agar plates. To maximize microbe pathogenicity for the porcine models, the Xen strain with strongest bioluminescence in the piglets was selected after a limited screen, and the porcine-passaged strain was cultured from a port abscess. All piglet experiments use this porcine primed Xen36 strain.

Mouse endocarditis

To induce mouse endocarditis, previous protocols for artery isolation surgery were followed, 4.0 suture material insertion and S. aureus infection as previously reported (Panizzi, P., et al., In vivo detection of Staphylococcus aureus endocarditis by targeting pathogen-specific prothrombin activation. Nat Med. 17(9), 1142-6 (2011)).

Mouse renal infection

Mice (n=14 mice) were anesthetized with isoflurane (1-3%/2L O2) and received 6×10⁷ CFU of S. aureus Xen 36 in 50 μl sterile phosphate buffered saline by intravenous injection. Mice were imaged for bioluminescence signal in the region of the kidneys starting at 48 hrs. post infection. Mice (n=8) showing apparent kidney infection were separated and half of those animals received the DAB-VT680XL (10 nmol) by intravenous injection. All mice were imaged at 24 hrs. after probe injection, when unbound DAB-VT680XL was excreted, and compared to non-infected control animals that only received the DAB-VT680XL injection (n=3). Imaging entailed collection of both bioluminescence (300s exposure time) and fluorescence (675 nm excitation and 720 nm emission filters) using an IVIS Lumina XRMS system (PerkinElmer Inc.). Mice were euthanized and confirmatory in situ and ex vivo images were also collected. for Auburn University.

Piglet endocarditis

A total of 36 newly weaned piglets (16-20 days old, weighing 10-15 lb) were purchased from the Swine Research and Education Center at Auburn University for use in model development (BLI only), fluorescence probe co-localization, and clinical PET/MRI studies. The animals were acclimated for 5-7 days prior to central-line implantation surgery. Piglets were sedated with dexmedetomidine (Dexdomitor; Zoetis, Parsippany, NJ) and butorphanol. An intravenous catheter was placed and anesthesia was induced using a combination of ketamine (10 mg/kg; Ketaset; Zoetis, Kalamazoo, MI), dexmedetomidine (20 mcg/kg) and butorphanol (0.4 mg/kg). A line block of 0.5% lidocaine (Xylocaine-MPF; Fresenius Kabi USA, Lake Zurich, IL) was placed prior to making a 3-4 cm incision just lateral to the midline. A combination of sharp and blunt dissection was used to identify and isolate the left jugular vein and then to create a subcutaneous pocket for the vascular access port (VAP; 5Fr ClearPort; Access Technologies; Skokie, IL). The vascular port consisted of a titanium outlet with a silicone septum and catheter. A small jugular venotomy was made and a 0.025 guide-wire was introduced into the vascular lumen. The polyurethane VAP catheter was placed over the guide-wire and advanced into the right ventricle under fluoroscopic guidance. Correct positioning of the catheter was confirmed using multiple injections of radiopaque contrast under fluoroscopic observation. Ports were implanted in the front right region of the neck. Once the desired catheter positioning was confirmed, the VAP catheter was secured within the jugular vein with several circumferential sutures of 3-0 polypropylene (Prolene; Ethicon, Summerville, NJ). The catheter tubing was cut to an appropriate length and connected to the VAP that was then secured within the subcutaneous pocket with multiple polypropylene sutures. The surgical site was lavaged with saline and closed with 3-0 poliglecaprone 25 (Monocryl; Ethicon, Summerville, NJ) in the subcutaneous and intra-dermal layers. A 22-gauge Posi-grip Huber point needle was placed into the VAP, continued patency was confirmed and the VAP was heparin-locked. The VAP site was marked with a permanent skin marker for ease of injection. The piglets then recovered from anesthesia. Analgesia was provided with carprofen (2.2 mg/kg PO; q12h; Rimadyl; Zoetis, Kalamazoo, MI) and butorphanol (0.2-0.4 mg/kg, IM, q4-6 h, PRN). At 6-8 hours following surgery, piglets were injected with 4-8×10⁸ CFU of S. aureus Xen 36 (PerkinElmer Inc.) through the VAP using a Huber needle. Thereafter the port was flushed with 5 mL sterile PBS.

For aortic valve endocarditis, piglets were similarly prepared but the aorta was accessed via the left carotid artery. Aortic valve damage was induced by repeated passing of a 2.5 mm diameter cytology brush (Endoscopy Support Services, Inc.; Brewster, NY) through the valve. The brush was positioned under fluoroscopy guidance aided by repeated contrast injection. A venous leg catheter used to administer anesthesia and the S. aureus Xen 36 inoculum (5-8×10⁸ CFU) followed by a bolus 60 mL sterile saline flush.

For optical studies, piglets were injected with 0.4 umol DAB-VT680XL in 2 mL sterile PBS via the ear vein using a 25-gauge butterfly. Animals were euthanized 10-12 hours later and BLI and FRI performed immediately following necropsy using an IVIS Lumina XRMS imaging system (PerkinElmer Inc.). For PET/MRI, piglets were transferred to Mt. Sinai Hospital.

Synthesizing fluorescent and fluorine-18 labeled dabigatran

The fluorescent and nuclear thrombin-specific imaging agents are derived from the FDA-approved thrombin inhibitor dabigatran. Synthesizing both agents requires converting the parent compound's carboxylic acid functionality to an amine, which can be further modified with either a fluorochrome or ¹⁸F-prosthetic group. Synthesizing Dabigatran-NH₂

Dabigatran (50 mg, 106 μmol) was suspended in dimethylformamide (DMF, 4.0 mL) in a 20-mL vial with a magnetic stir-bar, to which N-Boc-2, 2′-(ethylenedioxy)diethylamine (105 mg, 424 μmol) and EDC (265 mg, 1.38 mmol) were then added. After stirring for 3 h, the reaction mixture was concentrated to dryness, re-dissolved in DMSO:H₂O (2.0:0.1 mL) and subjected to reverse phase chromatography, resulting in 50 mg for a 67.2% isolated yield of Dabigatran-NH-Boc. LC-ESI-MS(+) m/z=702.5 [M+H+]+. Dabigatran-NH-Boc was dissolved in H₂O:MeCN (1:1, 400 uL), and then HCl (4 M) in dioxane (1 mL) was added. The homogeneous solution was stirred at room temperature for 30 min and the reaction was concentrated by rotovap to give 41 mg, a 95.6% yield, of Dabigatran-NH₂ as a colorless solid. LC-ESI-MS(+) m/z=602.4 [M+H+]+; LC−ESI−MS(−) m/z=600.4 [M−H+]−.

Synthesizing DAB-VT680XL

Dabigatran-NH₂ (0.5 mg, 0.7 μmol) was dissolved in DMF (12 μL) in a 1.5-mL centrifuge tube and added to VivoTag680 XL-NHS ester (1.0 mg, 0.7 μmol) in DMF (100 uL). After 3 h, this mixture was concentrated to dryness then redissolved in H₂O/MeCN (10:1, 110 uL) and subjected to C18 reverse-phase HPLC purification. The combined HPLC collections were concentrated to give 1.1 mg of product, a 75.6% yield. LC-ESI-MS(−) m/z=917.4 ([M-2H+]/2)−, m/z=611.2 ([M-3H+]/3)−.

Synthesizing ¹⁹F-DAB

Dabigatran-NH₂ (4 mg, 6.7 μmol) was dissolved in DMF (100 μL) and triethylamine (3 μL) in a 1.5-mL centrifuge tube and treated with N-succinimidyl-4-fluorobenoate (3 mg, 12.5 μmol) in DMF (50 μL). After 4 h the mixture was concentrated by rotary evaporation and subjected to HPLC purification resulting in 4.1 mg of ¹⁹F-Dabigatran, a 68% yield. LC-ESI-MS(+) m/z=724.6 [M+H+]+, 746.6 [M+Na+]+.

Synthesizing ¹⁸F-DAB

The prosthetic group N-succinimidyl-4-[¹⁸F]-fluorobenzoate (¹⁸F-FSB) was synthesized following the automated procedure of Scott and Shao (25), adapted for a Synthra RN Plus automated synthesizer (Synthra GmbH, Hamburg, Germany) operated by SynthraView software. Starting with [¹⁸F]-F−, n.c.a., (˜1772 MBq, 50±4 mCi), 18F-SFB was prepared in 25.0% isolated yield in 100 min. Dabigatran-NH₂ (4 mg, 6.7 umol) dissolved in acetonitrile (500 μL) and triethylamine (4 μL) was reacted with ¹⁸F-SFB (447 MBq, 12±3 mCi) at 65° C. for 5 min, cooled and subjected to C18 reverse-phase HPLC using a Machery-Nagel Nucleodur C18 Pyramid 250×10 mm Vario-Prep column eluted with 75:25 water-acetonitrile (100 mM ammonium formate) at 5.5 mL/min and a 254 nm UV detector and radiodetector connected in series. ¹⁸F-Dabigatran was synthesized in 10.7% isolated yield (189 MBq, 5.1±0.2 mCi) and at 99±0.9% radiochemical purity.

¹⁸F-Dabigatran for piglet imaging was produced using a GE FX2N automated synthesizer (GE Healthcare, Chicago, IL, USA). A QMA cartridge containing cyclotron-produced [¹⁸F]fluoride (˜30 GBq, 0.81±0.05 Ci) was eluted with a solution containing 9 mg 4,7,13, 16,21,24-hexaoxa-1,10 diazabicyclo[8.8.8]hexacosane (Kryptofix [2.2.2]); 0.08 mL 0.15 M K₂CO₃ and 1.92 mL acetonitrile into a 5 mL reaction vial. Solvents were removed azeotropically at 110° C.under a slight flow of helium. Then, N-succinimidyl-4-[¹⁸F]-fluorobenzoate (¹⁸F-SFB) was synthesized in 30% isolated yield (as described in previous section) and reacted with Dabigatran-NH₂ (4 mg, 6.7 μmol) dissolved in acetonitrile (500 μL) and triethylamine (4 μL) at 65° C. for 5 min. The reaction mixture was purified by HPLC using a C-18 semi-preparative column (Luna C-18, 250×10 mm, 5 μm -Phenomenex, Torrance, CA, USA) and isocratic elution with 90:10 water (75 mM ammonium formate)/ethanol at 5 mL*min⁻¹ and a 254 nm UV detector. ¹⁸F-Dabigatran was synthesized in 8±1.2% d.c. radiochemical yield (2.3±1.1 GBq, 0.06±0.03 Ci, RT=32 min) and at >98% radiochemical purity. Purity was assessed via Radio-HPLC using a C-18 analytical column (Atlantis T3, 100 Å, 250×4.6 mm, 5 μm-Waters, Milford, MA, USA, RT=9.8 min).

Thrombin activity assay

To confirm that modification did not inhibit binding activity, VT680XL and ¹⁹F-labeled dabigatran were examined with the SensoLyte AFC Thrombin Assay Kit (AnaSpec, Inc. Fremont, CA, USA). Thrombin cleaves the substrate, releasing 7-amido-4-trifluoromethylcoumarin, which was monitored at excitation/emission=380/500 nm.

¹⁸F-DAB blood half-life

To determine the blood half-life of the fluorine-18 (¹⁸F)-labeled imaging agent derived from the thrombin inhibitor dabigatran (¹⁸F-DAB), blood from ¹⁸F-DAB-injected mice was collected by retro-orbital bleeding and sampled with gamma-counting. Under isoflurane (1.5-3%) anesthesia, ¹⁸F-DAB was injected via tail vein (approximately 250 μCi in 100 μl PBS) in 12-week-old 6 C57BL/6 mice. Mice were kept on a heated stage (37° C.) under isoflurane anesthesia and bled 20 μl 1-2, 5, 10, 15, 30, 60 and 120 minutes after probe injection. Blood samples were weighed and residual radioactivity in the samples was measured using a gamma-counter and the percent injected dose per gram blood (%IDGB) was computed. Blood half-life was derived from fitting %IDGB to the one compartment pharmacokinetic equation C(t)=C(o)e^{kt} whereas C(t) is %IDGB at time t and k is the rate constant. Half-life is denoted as

$${t}_{1 / 2} = \frac{ln ( 2 )}{k} .$$

Intravital microscopy of thrombi in the femoral artery

Intravital microscopy was used to visualize DAB-VT680XL binding to freshly formed thrombi. Arterial thrombosis was induced by applying ferric chloride solution (500 mM concentration; Sigma) on the exposed femoral artery of mice. Fluorescently conjugated anti-CD41 mAb (Biolegend) was injected via tail vein to label platelets in vivo before thrombosis induction. DAB-VT680XL and control fluorochrome VT680XL were injected intravenously 5 min after thrombosis induction. Images were acquired with IVM (Olympus) in vivo.

Intravital microscopy of vegetation in the femoral artery

A 12-0 Ethicon™ suture material was inserted into the saphenous artery, advanced into the femoral artery and fixed in position while maintaining sufficient blood flow. Mice were allowed to recover for 6 hours before injection of 10⁶ CFU S. aureus ^{RFP+} bacteria in 100 μl PBS. 6 hours after bacteria administration, SC− and vWbp-neutralizing mAb (GMA-2105 and GMA-2510, Green Mountain Antibodies) or isotype IgG-control antibodies were injected. 90 minutes before imaging, neutrophils were labeled by injecting 15 μg FITC anti-mouse Ly-6G antibody (Clone 1A8, BioLegend) and the vegetation was stained by injecting 2 nmol DAB-VT680XL. All injections were done via tail vein. Intravital microscopy was performed 24 hours after bacteria injection. Mice were anesthetized using 1-2% isoflurane, then placed on a heated (37° C.) stage for imaging, and the wound was reopened. Imaging was done using an Olympus (IV100) microscope with a water-immersion objective (UMPlanFL N 20x NA 0.50, Olympus). Three channels were recorded (Ly-6G FITC, 488nm ex; RFP, 561 nm ex; DAB-VT680XL, 647 nm ex) to generate z-stacks at 2 μm steps. Image post-processing was performed using ImageJ software.

FMT/CT

On day 3 after suture insertion and 48 hours after injection of either 1×10⁶ CFU S. aureus Xen29 in 100 μl PBS or PBS only for the sham group, FMT/CT imaging was performed. To this end, mice were injected with 2 nmol of the fluorescent imaging probe and imaged 2 hours later using an FMT-2500 LX Quantitative Tomography Imaging System (PerkinElmer). After excitation at 680 nm and emission collection at 700 nm, a three-dimensional dataset containing fluorescence concentration per voxel was reconstructed. FMT imaging was accompanied by hybrid X-ray CT angiography (Inveon PET-CT, Siemens). Image fusion was achieved using Osirix software and fiducial markers on a dedicated multimodal imaging cassette frame, as described previously (26). During CT acquisition, IsoVue 370 was infused at 50 μl/min through a tail vein catheter. The CT was reconstructed using a modified Feldkamp cone beam reconstruction algorithm (COBRA, Exxim Inc.), bilinear interpolation and a Shepp-Logan reconstruction filter. Voxels were scaled to Hounsfield units. The isotropic spatial resolution was 110 μm for CT and 1 mm for FMT. Fused data sets were used to place regions of interest in the left ventricular outflow tract and the aortic valve region. After FMT-CT, underwent ex vivo fluorescence imaging of excised aortas on an OV-110 epifluorescence microscope (Olympus). The same setup was used to evaluate the effects of SC und vWbp-neutralizing mAb treatment. Six hours after bacteria injection, either SC und vWbp-neutralizing mAb or unspecific IgG-control antibodies were injected.

Fluorescence reflectance imaging and histology

Excised aortas were imaged side-by-side with controls using epifluorescence microscope (OV-110, Olympus). The tissue was then fixed in 4% paraformaldehyde (PFA) for at least 12 hours, embedded in optimal-cutting-temperature compound and flash-frozen in an isopentane/dry ice bath. Hematoxylin and eosin (H&E), Gram staining (Sigma-Aldrich) and immmunofluorescence staining for CD11b were performed to verify the presence of S. aureus bacteria and myeloid cells on the aortic valve. Fluorescence microscopy (Eclipse 80i, Nikon) was performed to investigate microscopic DAB-VT680XL localization in the vegetation, and bright field images were scanned and analyzed using a Nanozoomer 2.0RS (Hamamatsu, Japan).

PET/CT imaging in mice

On day 3 post surgery, animals were injected with 250 μCi of ¹⁸F-DAB and imaged by PET-CT 1.5 hours later. We used an Inveon small animal PET-CT scanner (Siemens), a 3D ordered subsets maximum likelihood with maximum a posteriori (OSEM3D/MAP) algorithm with 2 OSEM and 18 MAP iterations to reconstruct into three-dimensional images. The CT was performed prior to the PET scan. The PET voxel size was 0.796×0.861×0.861 mm, for a total of 128×128×159 voxels. Standard uptake values (SUV) were obtained from manually drawn regions of interest in the invent research workplace software environment. Following PET/CT imaging, the aortic root was excised, counted on a Wallac wizard 3 gamma counter to obtain percent injected dose per gram tissue (%IDGT) and imaged for bioluminescent signal. This was followed by overnight exposure on an autoradiography cassette. Plates were read on a Typhoon™ 9400 Variable Mode Imager (GE Healthcare, Chicago, IL, USA). Target to background of both the bioluminescent signal and autoradiography were quantified using manual ROI's of the aorta and background in Amira software (ThermoFisher Scientific).

Generating monoclonal antibodies that neutralize SC and vWbp

The murine monoclonal antibodies against synthetic peptides corresponded to the N-terminal residues 1 through 10 of either SC or vWbp from S. aureus Newman D2 Tager 104 strain. Corresponding peptides were synthesized with an additional C-terminal Cys that conjugated to keyhole limpet hemocyanin (KLH) and ovalbumin (OA) using m-maleimidobenzoyl-N-hydroxysuccinimide ester. To generate these monoclonal antibodies, mice were injected on day 1 with KLH-peptide conjugate (100 μg) in complete Freund's adjuvant. On days 17, 27 and 42, mice were injected with KLH-peptide conjugate (50 μg) in incomplete Freund's adjuvant. Serum titers from each mouse were determined by solid-phase ELISA, and spleen cells from the mouse with the highest serum titer were fused to NS1 myeloma cells on day 162, as described using polyethylene glycol. Hybridoma were selected using hypoxanthine, azaserine and thymidine. Fusion clones were screened by solid-phase ELISA with peptide-OA coated microtiter plates. Selected clones showing signal above ˜2x-background were expanded, re-screened, sub-cloned three times by limiting dilution and stored in liquid nitrogen. SC-specific antibodies were designated GMA-2105 and others specific for vWbp were designated GMA-2510. Hybridoma cells were grown in Hybridoma-SFM media (Gibco) and antibodies purified by protein G affinity chromatography. Purified antibody was sterile-filtered and stored at 4° C. Antibody aggregation was ruled out by size exclusion chromatography on an S-300 column and dynamic light scattering with a Zetasizer Nano-S instrument (Malvern Panalytical Ltd, Malvern, United Kingdom). The isotype of each respective antibody was independently verified using/via goat anti-mouse isotype-specific antibody (Bethyl Laboratories Inc., Montgomery, TX, USA) using a MagPix (Luminex Corp., Austin, TX, USA).

Antibody specificity for SC and vWbp

Western blot confirmed specificity of prothrombin activation-specific monoclonal antibodies. Previously characterized recombinant proteins were subjected to SDS gel electrophoresis with lanes corresponding to (1) vWbp-(1-263), (2) vWbp-(1-474), (3) SC-(1-325),(4) SC-(1-660) and (5) protein standards with indicated molecular weights. The elaborated proteins were transferred to PVDF membrane for western blot analysis to probe the specificity and cross-reactivity of the monoclonal antibodies targeting the critical N-termini of either SC or vWbp. The same blot was probed with either the anti-vWbp monoclonal antibody (GMA-2510) (5 μg/mL) or the anti-SC (GMA-2105) (10 μg/mL) for 1 hour at 4° C. Following primary antibody treatment, blots were washed and probed with horseradish peroxidase-labeled rabbit anti-mouse IgG that lacked the constant region and then imaged for chemiluminescence substrate oxidation using a Fuji-Films LAS1000. The blot was stripped between primary antibody challenges. Finally, since the antibodies were intended to be used together, we verified that GMA-2510 and GMA-2105 would have synergistic functions in recognizing these S. aureus virulence factors. To accomplish this, the blot was probed with the both the SC and vWbp neutralizing antibodies and then stained for total mouse IgG content using an anti-mouse IgG (H+L)-FITC polyclonal antibody. The blot was imaged for fluorescence using a Fuji-films FLA5100 with the 473 laser and LBP channel.

Fibrinogen turbidity assays

Cleavage of fibrinogen by either prothrombin.vWBp-(1-263) or prothrombin●SC(1-325) complexes was monitored from the increase in turbidity at 450 nm at 25° C. in 50 mM Hepes, 110 mM NaCl, 5 mM CaCl2, 1 mg/mL polyethylene glycol (PEG) 8000 (pH 7.4) buffer by using a SpectraMax 340 PC 384 plate reader (Molecular Devices Inc.). Individual reaction conditions were tested to determine the effect of the respective antibodies on the ability of either vWbp or SC to activate prothrombin and subsequently cleave of fibrinogen. GMA-2510 (anti-vWbp Ab) was incubated with vWbp-(1-263) and GMA-2105 (anti-SC Ab) was incubated with SC-(1-325) for 25 minutes at 25° C. prior to addition of prothrombin. The 3 components were then incubated together for an additional 25 minutes at 25° C. prior sub-sampling into the turbidity assay. The vWbp assays had final concentrations of 75nM prothrombin●vWBp(1-263) complex with either OnM, 300 nM or 1.5 μM anti-vWbp ab. The SC assays had 15 nM prothrombin●SC(1-325) complex with either OnM, 50 nM, or 300 nM anti-SC ab. Fibrinogen (1.5 mg/mL) was added simultaneously to initiate all reactions. Progress curves were collected over time ranges necessary to observe total substrate depletion under the positive control conditions.

Survival study

To determine the potentially beneficial impact of eliminating prothrombin activation by bacteria, we simultaneously administered either both GMA-2105 and 2510 mAbs or an isotype control mAb. Endocarditis was induced in 30 mice, which were randomized to treatment groups. Six hours post surgery, the mice received GMA-2105, GMA-2510 or isotype-labeled mAbs by intraperitoneal injection. Mice were kept under normal husbandry without further treatment except for pain management with buprenorphine as needed until death occurred, humane endpoints were reached or up to day 7 after injection of the 1×10⁶ CFU S. aureus.

MRI of piglets

Left ventricular ejection fraction was quantified from retrospectively gated short-axis cardiac cine MR images (Siemens 3T Biograph mMR). Acquisition parameters for cine short axis stacks were as follows: repetition time (TR) 56.24 ms, echo time (TE) 3.32 ms, number of averages 2, 24 or 30 slices, 25 cardiac frames, 3 mm slice thickness, no interslice gap, flip angle 12, spatial resolution 0.94×0.94 mm². Retrospective ECG gating was used to acquire the images. ROls were manually segmented with Osirix MD v 9.5.1 and exported using the ‘Export ROIs’ Osirix plugin. The cine acquisition contains a total of 600 or 750 images from 24 slices with 25 cardiac frames per slice. Right ventricle vegetations were quantified from an ECG triggered axial T2 weighted turbo spin echo (TSE) stack using the following acquisition parameters: TR 1125-1485 ms, TE 76 ms, number of averages 4, 11-24 slices, 3 mm slice thickness, no interslice gap, spatial resolution 0.94×0.94 mm². ROIs were manually segmented with Osirix MD v 9.5.1. ROls were exported using the ‘Export ROIs’ Osirix plugin. Vegetations were segmented as high intensity areas within the right ventricle while excluding the catheter whenever possible.

PET/MRI of piglets

Eight piglets underwent imaging with a clinical PET/MR system (Siemens 3T Biograph mMR). The piglets received an intravenous injection of ¹⁸F-DAB (51.8 and 25 MBq, respectively) 90 minutes before PET acquisition. Piglets were intubated and placed on the scanner bed under isoflurane anesthesia at 1.5-2% by inhalation, and were oxygenated throughout the PET/MR imaging experiment. Vital parameters were monitored. A 6-channel body matrix product coil was used for signal reception. Following scout scans, a static thoracic PET was performed for 60 minutes while simultaneously acquiring cardiac and T2 weighted TSE anatomical MR images as detailed above. Attenuation correction of PET images was performed by using a vendor-built-in Dixon MR-based attenuation map (MR-AC) with 4 tissue compartments (soft tissue, fat, lung and air). Images were reconstructed using a 3D ordinary Poisson ordered subsets expectation maximization (OP-OSEM) algorithm with point-spread-function (PSF) resolution modeling, using 3 iterations and 21 subsets and filtered with a 4 mm Gaussian filter.

Autoradiography of piglet samples

Following euthanasia, animals were perfused and heart samples were excised. To determine radiotracer distribution, digital autoradiography was performed by placing tissue samples in a film cassette against a phosphorimaging plate (BASMS-2325, Fujifilm, Valhalla, NY, USA) for 12.5 hours at −20° C. Phosphorimaging plates were read at a pixel resolution of 25 μm with a Typhoon 7000IP plate reader (GE Healthcare, Pittsburgh, PA, USA). Quantification was carried out using ImageJ software.

Statistical analysis

Results are reported as mean ±standard error of mean (SEM). Statistical analysis was performed using GraphPad Prism 7 software (GraphPad Software, Inc.). Normal distribution of variables was tested using the Kolmogorov-Smirnov-test or the D'Agostino-Pearson omnibus normality test. Data were analyzed by parametric tests if normal distribution was detected. An unpaired student t-test was applied for two-group comparisons and data presented as mean ±s.e.m. with significance indicated by *P<0.05, **P<0.01, ***P<0.001 and **** P<0.001. If more than two groups were compared, one-way analysis of variance (ANOVA) analysis and Bartlett's test for equal variances was used. If data were non-normally distributed, differences were evaluated using an unpaired, nonparametric Mann-Whitney test. A log-rank test was applied in the survival study. Significance level in all tests was 0.05

Therapeutic Applications

Monoclonal antibodies (mAbs) that specifically inhibit staphylocoagulase and or vWbp functions are less likely than anti-coagulants to have off-target adverse effects and would be a significant step forward.

The monoclonal antibodies disclosed herein are usable as inhibitors of staphylocoagulase and/or vWbp.

The antibodies provided may also be formed into suitable pharmaceutical compositions, for administration to a human or animal patient in order to treat or prevent an infection caused by staphylococcal bacteria. Pharmaceutical compositions containing the antibodies provided, variations, and/or effective fragments thereof, may be formulated in combination with any suitable pharmaceutical vehicle, excipient or carrier that would commonly be used in this art, including such conventional materials for this purpose, e.g., saline, dextrose, water, glycerol, ethanol, other therapeutic compounds, and combinations thereof. The particular vehicle, excipient or carrier used will vary depending on the patient and the patient's condition, and a variety of modes of administration would be suitable for the compositions, as would be recognized by one of ordinary skill in this art. Suitable methods of administration of any pharmaceutical composition disclosed in this application include, but are not limited to, topical, oral, anal, vaginal, intravenous, intraperitoneal, intramuscular, subcutaneous, intranasal and intradermal administration.

If topical administration is desired, the composition may be formulated as needed in a suitable form, e.g., an ointment, cream, gel, lotion, drops (such as eye drops and ear drops), or solution (such as mouthwash). Wound or surgical dressings, sutures and aerosols may be impregnated with the composition. The composition may contain conventional additives, such as preservatives, solvents to promote penetration, and emollients. Topical formulations may also contain conventional carriers such as cream or ointment bases, ethanol, or others.

The antibody compositions provided will thus be useful for interfering with, modulating, or inhibiting binding interactions between the staphylococcal-secreted staphylocoagulase and/or vWbp protein and its ligand protein prothrombin in blood and tissues, and will thus have particular applicability in developing compositions and methods of preventing or treating staphylococcal infection, and in inhibiting the activation of prothrombin.

Methods are provided for preventing or treating a staphylococcal infection which comprise administering an effective amount of the monoclonal antibody as described above in amounts effective to treat or prevent the infection. In addition, these monoclonal antibodies (in various forms including murine, chimeric, humanized sequences, and F(ab) fragments), and have been shown to have high affinity in binding of staphylocoagulase and vWbp secreted by staphylococcal bacteria, and effective in treating or preventing infection from staph bacteria such as S. aureus. This tendency is supported by the mouse survival data provided herein. Further, as demonstrated by the inhibition studies, these monoclonals will be useful in inhibiting S. aureus from activating host coagulation mechanisms, e.g., those involved in development of AIE.

Accordingly, administration of the antibodies disclosed herein in any of the conventional ways described above (e.g., topical, parenteral, intramuscular, etc.), may provide an extremely useful method of treating or preventing staphylococcal infections in human or animal patients. By effective amount is meant that level of use, such as of an antibody titer, that will be sufficient to either prevent adherence of the bacteria, to inhibit binding of staph bacteria to host cells and thus be useful in the treatment or prevention of a staph infection. As would be recognized by one of ordinary skill in this art, the level of antibody titer needed to be effective in treating or preventing staphylococcal infection will vary depending on the nature and condition of the patient, and/or the severity of the pre-existing staphylococcal infection.

In addition to the use of disclosed antibodies and degenerative or homologs thereof to treat or prevent S. aureus infection as described above, we contemplate the use of these antibodies in a variety of ways, including the detection of the presence of S. aureus to diagnose a staph infection, whether in a patient or on medical equipment, implants or prosthetics which may also become infected. For example, a method of detecting the presence of staph infections involves the steps of obtaining a sample suspected of being infected by one or more staphylococcal bacteria species or strains, such as a sample taken from an individual, for example, from one's blood, saliva, tissues, bone, muscle, cartilage, or skin. The cells can then be lysed, and the DNA extracted, precipitated and amplified. Following isolation of the sample, diagnostic assays utilizing the disclosed antibodies may be carried out to detect the presence of S. aureus, and such assay techniques for determining such presence in a sample are well known to those skilled in the art and include methods such as radioimmunoassay, Western blot analysis and ELISA assays. A method of diagnosing an S. aureus infection is contemplated wherein a sample suspected of being infected with S. aureus infection has added to it the monoclonal antibody described herein, and S. aureus is indicated by antibody binding to the staphylocoagulase and/or vWbp proteins in the sample.

Accordingly, disclosed antibodies may be used for the specific detection or diagnosis of staphylococcal proteins, for the prevention of infection from staph bacteria, for the treatment of an ongoing infection, or for use as research tools. The term “antibodies” as used herein includes monoclonal, polyclonal, chimeric, single chain, bispecific, simianized, and humanized or primatized antibodies as well as F(ab) fragments, such as those fragments which maintain the binding specificity of the antibodies to the staphylocoagulase and/or vWbp proteins, including the products of an F(ab) immunoglobulin expression library. Accordingly, we contemplate the use of single chains such as the variable heavy and light chains of the antibodies as will be set forth below. Generation of any of these types of antibodies or antibody fragments is well known to those skilled in the art. In the present case, monoclonal antibodies to staphylocoagulase and/or vWbp proteins have been generated against N-terminal staphylocoagulase protein and have been isolated and shown to have high affinity to S. aureus. Moreover, the monoclonals provided have been shown to recognize a high number of strains, on an equivalent level to that recognize by polyclonal antibodies to staphylocoagulase and/or vWbp, and thus can be used effectively in methods to protect against staphylococcal infection or treat same.

Antibodies to staphylocoagulase and/or vWbp as described above may also be used in production facilities or laboratories to isolate additional quantities of the proteins, such as by affinity chromatography. For example, the antibodies may also be utilized to isolate additional amounts of the staphylocoagulase and/or vWbp proteins or their active fragments.

The isolated antibodies provided herein, or active fragments thereof, may also be utilized in the development of vaccines for passive immunization against staph infections. Further, when administered as pharmaceutical composition to a wound or used to coat medical devices or polymeric biomaterials in vitro and in vivo, the antibodies may be useful in those cases where there is a previous staph infection because of the ability of this antibody to further restrict and inhibit S. aureus staphylocoagulase and/or vWbp binding to prothrombin and thus limit the extent of the infection. In addition, the antibody may be modified as desired so that, in certain instances, reduce the immunogenicity in the patient to whom it is administered. For example, if the patient is a human, the antibody may be “humanized” by transplanting the complementarity determining regions (CDR's) (e.g., SEQ ID Nos. 10-15) of the hybridoma-derived antibody into a human monoclonal antibody as described, e.g., by Jones et al., Nature 321:522-525 (1986) or Tempest et al. Biotechnology 9:266-273 (1991) and demonstrated herein. They may also be “veneered” by changing the surface exposed murine framework residues in the immunoglobulin variable regions to mimic a homologous human framework counterpart as described, e.g., by Padlan, Molecular Imm. 28:489-498 (1991) and U.S. Pat. No. 6,797,492, all of these references incorporated herein by reference. Even further, when so desired, the disclosed monoclonal antibodies may be administered in conjunction with a suitable antibiotic to further enhance the ability of the present compositions to fight bacterial infections.

The antibodies may also be used as a passive vaccine which will be useful in providing suitable antibodies to treat or prevent a staphylococcal infection. As would be recognized by one skilled in this art, a vaccine may be packaged for administration in a number of suitable ways, such as by parenteral (i.e., intramuscular, intradermal or subcutaneous) administration or nasopharyngeal (i.e., intranasal) administration. One such mode is where the vaccine is injected intramuscularly, e.g., into the deltoid muscle, however, the particular mode of administration will depend on the nature of the bacterial infection to be dealt with and the condition of the patient. The vaccine may be combined with a pharmaceutically acceptable carrier to facilitate administration, and the carrier is usually water or a buffered saline, with or without a preservative. The vaccine may be lyophilized for resuspension at the time of administration or in solution.

While “SC” and “staphylocoagulase” is used throughout, it is contemplated that the disclosed monoclonal antibodies are also useful against homologs or degenerate versions of SC. While “vWbo” and “von Willebrand factor binding protein” is used throughout, it is contemplated that the disclosed monoclonal antibodies are also useful against homologs or degenerate versions of vWbp. Similarly, while DNA and amino acid sequences are disclosed, it is contemplated that the claims cover the disclosed sequences as well as substantially similar sequences.

Two DNA sequences are “substantially similar” when approximately 70% or more (e.g., at least about 80%, at least about 89%, 90%, 91%, 92%, 93%, 94%, 95%, 96%, 97%, 98%, 99%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., Molecular Cloning: A Laboratory Manual, 1982; DNA Cloning, Vols. I & II, supra; Nucleic Acid Hybridization, [B. D. Hames & S. J. Higgins eds. (1985)].

By “substantially similar” is further meant a DNA sequence which, by virtue of the degeneracy of the genetic code, is not identical with that shown in any of the sequences disclosed, but which still encodes the same amino acid sequence; or a DNA sequence which encodes a different amino acid sequence that retains the activities of the proteins, either because one amino acid is replaced with a similar amino acid, or because the change (whether it be substitution, deletion or insertion) does not affect the active site of the protein.

Two amino acid sequences or two nucleic acid sequences are “substantially similar” when approximately 70% or more (e.g., at least about 80%, at least about 89%, 90%, 91%, 92%, 93%, 94%, 95%, 96%, 97%, 98%, 99%) of the amino acids match over the defined length of the sequences.

As demonstrated by evidence herein, modification and changes may be made in the structure of the peptides and DNA segments which encode them and still obtain a functional molecule that encodes a protein or peptide with desirable characteristics. The changing the amino acids of a protein may be used to create an equivalent, or even an improved, second generation molecule.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, J. Mol Biol, 157(1):105-132, 1982.

It is known in the art that certain amino acids may be substituted by other amino acids having a similar hydropathic index or score and still result in a protein with similar biological activity, i.e., still obtain a biological functionally equivalent protein. In making such changes, there is a general trend toward substitution of amino acids whose hydropathic indices are within about ±0.5 to about ±2. It is also understood in the art that the substitution of like amino acids can be made effectively on the basis of hydrophilicity. For example, local average hydrophilicity of a protein, as governed by the hydrophilicity of its adjacent amino acids, may correlate with a biological property of the protein.

It is understood that an amino acid can be substituted for another having a similar hydrophilicity value and still obtain a biologically equivalent, and in particular, an immunologically equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include but are not limited to: arginine and lysine; glutamate and aspartate; serine and threonine; glutamine and asparagine; and valine, leucine and isoleucine.

The polypeptides can be chemically synthesized. The synthetic polypeptides are prepared using the well-known techniques such as but not limited to solid phase, liquid phase, or peptide condensation techniques, or any combination thereof, and can include natural and unnatural amino acids.

The invention is not limited to the embodiments illustrated and described, as it also covers all equivalent implementations insofar as they do not depart form the spirit of the invention. Further, the invention is not yet limited to the combination of features as described herein but may be defined by any other combination of all of the individual features disclosed. Further, the invention is not yet limited to the sequence of method steps as described herein but may be defined by any other combination or order the steps disclosed. Any person skilled in the art of will recognize from the previous detailed description and from the figures and claims that modifications could be made to the disclosed embodiments of the invention without departing from the scope of the invention.

1-20. (canceled)

21. An isolated monoclonal antibody or fragment thereof which binds an epitope of vWbp that is within SEQ ID. No: 4 and which comprises the following CDRs:
CDRH1:
GYSFTSYWIH
(Residues 26-35 of SEQ ID NO.: 29);
CDRH2:
AISPGNSDTNYNQNFKG
(Residues 50-66 of SEQ ID NO.: 29);
CDRH3:
ATGSHY
(Residues 97-102 of SEQ ID NO.: 29);
CDRL1:
QSLLDSDGTTY
(Residues 27-37 of SEQ ID NO.: 31);
CDRL2:
LVS
(Residues 55-57 of SEQ ID NO.: 31);
and
CDRL3:
WQGTHFPRT
(Residues 94-102 of SEQ ID NO.: 31).

22. A therapeutic agent comprising:
the isolated monoclonal antibody or fragment thereof of claim 2, and further comprising:
an isolated monoclonal antibody or fragment thereof which binds an epitope of SC that is within SEQ ID. No: 2 and which comprises the following CDRs:
CDRL1:
QNVDIY
(residues 27-32 of SEQ ID No. 8);
CDRL2:
SAS
(residues 50-52 of SEQ ID NO.: 8);
CDRL3:
QQYNNYPYT
(residues 89-97 of SEQ ID NO.: 8);
CDRH1:
GFTFSDAW
(residues 26-33 of SEQ ID NO.: 6);
CDRH2:
IRTKANNHAT
(residues 51-60 of SEQ ID NO.: 6);
and
CDRH3:
CTNVYYGNNDVKDY
(residues 98-111 of SEQ ID NO.: 6).

23. The isolated monoclonal antibody or fragment thereof of claim 21, wherein the isolated monoclonal antibody or fragment thereof is humanized.

24. The therapeutic agent of claim 22, wherein the isolated monoclonal antibody or fragment thereof is humanized.

25. A method of detecting the presence of S. aureus in a sample, said method comprising using an isolated monoclonal antibody or antigen-binding fragment thereof which binds an epitope of at vWbp peptide that is withing SEQ ID. NO.:4 and which comprises the following CDRs:
CDRH1:
GYSFTSYWIH
(Residues 26-35 of SEQ ID NO.: 29);
CDRH2:
AISPGNSDTNYNQNFKG
(Residues 50-66 of SEQ ID NO.: 29);
CDRH3:
ATGSHY
(Residues 97-102 of SEQ ID NO.: 29);
CDRL1:
QSLLDSDGTTY
(Residues 27-37 of SEQ ID NO.: 31);
CDRL2:
LVS
(Residues 55-57 of SEQ ID NO.: 31);
and
CDRL3:
WQGTHFPRT
(Residues 94-102 of SEQ ID NO.: 31).


SEQUENCE LISTING (41 sequences)

SEQ ID NO: 1 - DNA, 2067 bp, Staphylococcus aureus
atagtaacaaaggattatagcaaagagtcaagagtgaatgagaacagtaaatatgggacattaatttcagactggtatttaaaagggagattaactagtctagaatctcaatttatcaatgcattggatattttagagacatatcattatggcgaaaaagagtataaagatgcaaaagataaattgatgacaagaattttaggggaagaccaataccttttagaaagaaaaaaagtgcagtatgaggaatacaaaaaattataccaaaaatataaagaagagaatccaacctctaaagttaaaatgaaaacattcgatcaatatacaatagaagatttaactatgagggaatataatgagttaacagaatcattaaaaagtgctgtaaaagactttgagaaagatgttgaaaaaatagaaaatcaacatcatgatttgaaaccatttactgatgaaatggaagagaaggctacttctagagttgatgatttagcaaataaagcatatagtgtttattttgcatttgttagggatacacaacataaaactgaggcattagagttaaaagcgaaagtagatttagttttaggtgatgaggataaaccgcatcgtatttctaatgaaagaattgaaaaagaaatgataaaagatttagaatctattattgaagatttctttatagaaactggtttaaataagcctggtaatattacgagttatgatagtagtaaacatcactataaaaatcacagtgaaggttttgaggctctagtcaaagaaacaagagaagcagtagcaaacgctgacgaatcttggaaaactaaaactgtaaaaaaatacggtgaatctgaaacaaaatctcctgttgtaaaagaagagaacaaagttgaagaccctcaatcacctaaatttgataaccaacaagaggttaaaactacggctggtaaagctgaagaaacaacacaaccagttgcacaaccattagttaaaattccacagggcacaattacaggtgaaattgtgaaaggtccggaatatccaacgatggaaaataaaacgttacaaggtgaaatcgttcaaggtccagatttcccaacaatggaacaaagcggtccatctttaagcgacaattatactcaaccgacgacaccgaaccctattttagaaggtcttgaaggtagctcatctaaacttgaaataaaaccacaaggtactgaatcaacgttgaaaggtattcaaggagaatcaagtgatattgaagttaaacctcaagcaactgaaacaacagaagcttctcaatatggtccgagaccgcaatttaacaaaacacctaagtatgtgaaatatagagatgctggtacaggtattcgtgaatacaacgatggaacatttggatatgaagcgagaccaagattcaacaagccatcagaaacaaacgcatacaacgtaacgacaaatcaagatggcacagtatcatacggcgcccgcccaacacaaaacaaggcatcagaaacaaacgcatataacgtaacaacacatgcaaacggccaagtatcatacggagctcgcccaacacaaaagaagccaagcgaaacaaatgcatataacgtaacaacacatgcaaacggccaagtatcatatggcgcccgcccgacatacaacaagccaagcgaaacaaatgcatataacgtaacaacacacggaaatggccaagtatcatatggagctcgtccgacatacaagaaaccaagtaaaacaaatgcatataacgtaacaacacatgcaaacggccaagtgtcatacggagctcgcccaacacaaaagaagccaagcgaaacaaacgcatataacgtaacaacacatgcaaatggccaagtatcatacggagctcgcccaacacaaaagaagccaagcgaaacaaacgcatataacgtaacaacacacggaaacggtcaagtgtcatacggcgctcgtccgacatacaacaagccaagtaaaacaaatgcatacaatgtaacaacacatgcagatggtactgcgacatatggtcctagagtaacaaaataa

SEQ ID NO: 2 - protein, 689 aa, Staphylococcus aureus
IVTKDYSKESRVNENSKYGTLISDWYLKGRLTSLESQFINALDILETYHYGEKEYKDAKDKLMTRILGEDQYLLERKKVQYEEYKKLYQKYKEENPTSKGLKLKTFDQYTIEDLTMREYNELTESLKSAVKDFEKDVEKIENQHHDLKPFTDEMEEKATSRVDDLANKAYSVYFAFVRDTQHKTEALELKAKVDLVLGDEDKPHRISNERIEKEMIKDLESIIEDFFIETGLNKPGNITSYDSSKHHYKNHSEGFEALVKETREAVANADESWKTKTVKKYGESETKSPVVKEENKVEDPQSPKFDNQQEVKTTAGKAEETTQPVAQPLVKIPQGTITGEIVKGPEYPTMENKTLQGEIVQGPDFPTMEQSGPSLSDNYTQPTTPNPILEGLEGSSSKLEIKPQGTESTLKGIQGESSDIEVKPQATETTEASQYGPRPQFNKTPKYVKYRDAGTGIREYNDGTFGYEARPRFNKPSETNAYNVTTNQDGTVSYGARPTQNKASETNAYNVTTHANGQVSYGARPTQKKPSETNAYNVTTHANGQVSYGARPTYNKPSETNAYNVTTHGNGQVSYGARPTYKKPSKTNAYNVTTHANGQVSYGARPTQNKPSETNAYNVTTHANGQVSYGARPTQNKPSETNAYNVTTHGNGQVSYGARPTYNKPSKTNAYNVTTHADGTATYGPRVTK

SEQ ID NO: 3 - DNA, 873 bp, Staphylococcus aureus
gtagtaccagaaacaggtattaataaaataattccagattatgataaatataagaatgcactaaagctaaatgtgagtagtttaactaacaacaataactttgtagcttctgaagataaattgaaaaaaattgcagatccatcagcagctggtaaaattgtaggtggaaaatttgccgtactagaatcaaagttaggaagtattgtaccagagtacaaagaaataaataaacgtgcgaatgtaacaggaaacaacaatcccagtcataatattggaaagtcttttgttactaaaggtccagaagtaaaaagatttattacacaaaacaaagtaaaataccacttcattactacacaaacacactacaagaaagaagttacttcattcaaatcaacgcatgtacataaatatataaatcatgcaactacttctagcaataaacattttactgttaaaccaatagaagcgcctagatataaacacccatctcaatctttaattataaatcatcatattgcagtacctggctaccatgctcataaatttgtaacaccaggacatgctagtattaaaattcatcatttttgtattgcaccaaaaataaatagttttaaagtaattccatcatatggtcacagttcacaccgcatgcatgtaccaagttttcaaagtaatacaaaatcagtacatcaaaattctagagtaaataaagtatataactataaatacttctactcttataaagtagtgaaaggtgtgaagaaatattactcattttcaaaatcaaatgcttataaatttgttaaaccaccatttaatatcaaaaatgtaaattaccaatatgctgctttaagtaatagccctacacactaa

SEQ ID NO: 4 - protein, 481 aa, Staphylococcus aureus
VVSGEENPYVSKAIELKGTSNKSNTYENYRESLENLIFSLSFADYEKYEEPEYNNAVKKYQQKFMAEDDALKTFLSEEKKLEKTDRSRNSNGLLGLTHERYTYIFDTLKKNKQEFLQEIEEINLKNSDLKDFNDTEQYNADVEINNLENKVLMLGYTFFSTYKDEVEELYSELDLIVGEVQDKSDKKRAVNQRMLSRKKEDLESIIDKFFKEIKQERPENIPALTSDKNHNQSMALKLKSDTEAAKKDESNRSSRSKKSLDSQNYKSVSQEVTAEQKAEYEKRAEERKARFLDRQKSKKEPVVSLEYDFEHKQSVDNENDKQLVVSEPTKNPTLPTYIETTTQVPMPTVERQTQQQIIYKAPKQLAGLNGESHDFSTTHQTPTTSNHTHNNVVEFEETSALPGRKTGSLVGLSQIDSSHLTEREKRVIKREHVREAQKLVENYKDTHSYKDRLNAQQKVNTLSEGHQKRFNKQINKVYNGK

SEQ ID NO: 5 - DNA, 366 bp, Artificial Sequence
  Description of Artificial Sequence: Synthetic heavy chain variable region polynucleotide
gaagtgaagcttgaggagtctggaggaggcttggtgcaacctggaggatccatgaaactctcttgtgctgcctctggattcacttttagtgacgcctggatggactgggtccgccagtctccagagaaggggcttgagtgggttgctgaaattagaaccaaagctaataatcatgcaacatactatgctgagtctgtgaaagggaggttcaccatctcaagagatgattccaaaagcagtgtctacctgcaaatgaacagcttaagagctgaagacactggcatttattactgtaccaacgtctactatggtaacaacgatgttaaggactactggggtcaaggaacctcagtcaccgtctcccca

SEQ ID NO: 6 - protein, 124 aa, Artificial Sequence
  Description of Artificial Sequence: Synthetic heavy chain variable region polypeptide
EVKLEESGGGLVQPGGSMKLSCAASGFTFSDAWMDWVRQSPEKGLEWVAEIRTKANNHATYYAESVKGRFTISRDDSKSSVYLQMNSLRAEDTGIYYCTNVYYGNNDVKDYWGQGTSVTVSPAS

SEQ ID NO: 7 - DNA, 378 bp, Artificial Sequence
  Description of Artificial Sequence: Synthetic light chain variable region polynucleotide
gatattgtgatgacccagtctcaaaaattcatgtccacatcagtaggagacagggtcagcgtcacctgcaaggccagtcagaatgtggatatttatgtagcctggtatcaacagaaaccagggcaatctcctaaagcactgatttactcggcatcctaccggttcagtggagtccctgatcgcttcacaggcagtggatctgggacagatttcactctcagcatcagcaatgtgcagtctgaagacttggcagagtatttctgtcagcaatataacaactatccgtatacgttcggaggggggaccaagctggaaataaaacgggctgatgctgcaccaactgtatccatcttcccaccatccagtgaagggcgaatt

SEQ ID NO: 8 - protein, 126 aa, Artificial Sequence
  Description of Artificial Sequence: Synthetic light chain variable region polypeptide
DIVMTQSQKFMSTSVGDRVSVTCKASQNVDIYVAWYQQKPGQSPKALIYSASYRFSGVPDRFTGSGSGTDFTLSISNVQSEDLAEYFCQQYNNYPYTFGGGTKLEIKRADAAPTVSIFPPSSEGRI

SEQ ID NO: 9 - protein, 10 aa, Artificial Sequence
  Description of Artificial Sequence: Synthetic peptide
IVTKDYSKES

SEQ ID NO: 10 - protein, 6 aa, Artificial Sequence
  Description of Artificial Sequence: Synthetic CDRL1 peptide
QNVDIY

SEQ ID NO: 11 - protein, 3 aa, Artificial Sequence
  Description of Artificial Sequence: Synthetic CDRL2 peptide
SAS

SEQ ID NO: 12 - protein, 9 aa, Artificial Sequence
  Description of Artificial Sequence: Synthetic CDRL3 peptide
QQYNNYPYT

SEQ ID NO: 13 - protein, 8 aa, Artificial Sequence
  Description of Artificial Sequence: Synthetic CDRH1 peptide
GFTFSDAW

SEQ ID NO: 14 - protein, 10 aa, Artificial Sequence
  Description of Artificial Sequence: Synthetic CDRH2 peptide
IRTKANNHAT

SEQ ID NO: 15 - protein, 14 aa, Artificial Sequence
  Description of Artificial Sequence: Synthetic CDRH3 peptide
CTNVYYGNNDVKDY

SEQ ID NO: 16 - protein, 102 aa, Artificial Sequence
  Description of Artificial Sequence: Synthetic humanized GMA-2105 Light chain variable region version 1 polypeptide
MTQSPDSLAVSLGERATINCKASQNVDIYVAWYQQKPGQPPKALIYSASYRFSGVPDRFSGSGSGTDFTLTISSLQAEDVAVYYCQQYNNYPYTFGQGTKVE

SEQ ID NO: 17 - protein, 102 aa, Artificial Sequence
  Description of Artificial Sequence: Synthetic humanized GMA-2105 Light chain variable region version 2 polypeptide
MTQSPDSLAVSLGERATINCKASQNVDIYVAWYQQKPGQSPKALIYSASYRFSGVPDRFSGSGSGTDFTLTISSLQAEDVAVYYCQQYNNYPYTFGQGTKVE

SEQ ID NO: 18 - protein, 116 aa, Artificial Sequence
  Description of Artificial Sequence: Synthetic humanized GMA-2105 Heavy chain variable region version 1 polypeptide
LLESGGGLVQPGGSLRLSCAASGFTFSDAWMDWVRQAPGKGLEWVAEIRTKANNHATYYAESVKGRFTISRDDSKNTLYLQMNSLRAEDTAVYYCTNVYYGNNDVKDYWGQGTLVT

SEQ ID NO: 19 - protein, 116 aa, Artificial Sequence
  Description of Artificial Sequence: Synthetic humanized GMA-2105 Heavy chain variable region version 2 polypeptide
LLESGGGLVQPGGSLRLSCAASGFTFSDAWMDWVRQAPGKGLEWVAEIRTKANNHATYYAESVKGRFTISRDDSKSSVYLQMNSLRAEDTAVYYCTNVYYGNNDVKDYWGQGTLVT

SEQ ID NO: 20 - protein, 98 aa, Artificial Sequence
  Description of Artificial Sequence: Synthetic IgG1 CH1 polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 21 - protein, 15 aa, Artificial Sequence
  Description of Artificial Sequence: Synthetic IgG1 hinge region peptide
EPKSCDKTHTCPPCP

SEQ ID NO: 22 - protein, 110 aa, Artificial Sequence
  Description of Artificial Sequence: Synthetic IgG1 CH2 polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 23 - protein, 107 aa, Artificial Sequence
  Description of Artificial Sequence: Synthetic IgG1 CH3 polypeptide
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 107 aa, Artificial Sequence [sequence omitted]

SEQ ID NO: 25 - protein, 10 aa, Artificial Sequence
  Description of Artificial Sequence: Synthetic peptide
IVTKDYSAES

SEQ ID NO: 26 - protein, 10 aa, Artificial Sequence
  Description of Artificial Sequence: Synthetic peptide
IVTKDYSKAS

SEQ ID NO: 27 - protein, 10 aa, Artificial Sequence
  Description of Artificial Sequence: Synthetic peptide
IVTKDYSAAS

SEQ ID NO: 28 - protein, 12 aa, Artificial Sequence
  Description of Artificial Sequence: Synthetic peptide
VVSGEKNPYVSK

SEQ ID NO: 29 - protein, 113 aa, Artificial Sequence
  Description of Artificial Sequence: Synthetic GMAB-2500 Variable Heavy Chain Sequence (2500)
EVQLQQSGTVLARPGASVKMSCKTSGYSFTSYWIHWVKQRPGQGLEWIGAISPGNSDTNYNQNFKGKAKLTAVTSASTAYMELSSLTTEDSAVYYCATGSHYWGQGTTLSVSS

SEQ ID NO: 30 - DNA, 339 bp, Artificial Sequence
  Description of Artificial Sequence: Synthetic GMAB-2500 Variable Heavy Chain nucleotide sequence
gaggttcagctccagcagtctgggactgtgctggcaaggcctggggcttccgtgaagatgtcctgcaagacttctggctacagctttaccagctactggatacactgggtgaaacagaggcctggacagggtcttgaatggattggtgctatttctcctggaaatagtgatactaactacaaccagaatttcaagggcaaggccaaactgactgcagtcacatccgccagcactgcctacatggagctcagcagcctgacaactgaggactctgcggtctattactgtgctactggtagccactactggggccaaggcaccactctctcagtctcctca

SEQ ID NO: 31 - protein, 112 aa, Artificial Sequence
  Description of Artificial Sequence: Synthetic GMAB 2500 Light Chain Amino Acid Sequence
DVVMTQTPLTLSVTIGQPASISCKSSQSLLDSDGTTYLNWLLQRPGQSPKRLIYLVSKLDSGVPDRFTGSGSGTDFTLKISRVEAEDLGVYYCWQGTHFPRTFGGGTKLEIK

SEQ ID NO: 32 - DNA, 336 bp, Artificial Sequence
  Description of Artificial Sequence: Synthetic GMAB 2500 Light Chain nucleotide sequence
gatgttgtgatgacccagactccactcactctgtcggttaccattggacaaccagcctccatctcttgcaagtcaagtcagagcctcttagatagtgatggaacgacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcggacgttcggtggaggcaccaagctggaaatcaaa

SEQ ID NO: 33 - protein, 324 aa, Artificial Sequence
  Description of Artificial Sequence: Synthetic GMAB 2500 Constant Heavy Chain Amino Acid Sequence
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 107 aa, Artificial Sequence
  Description of Artificial Sequence: Synthetic GMAB 2500 Constant Light Chain Amino Acid Sequence
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 35 - protein, 113 aa, Artificial Sequence
  Description of Artificial Sequence: Synthetic GMAB 2510 Heavy Chain Amino Acid Sequence
QEQLQQSETALVKPGASVKMSCKASGFTFTNYFMHWVKQTPGQGLEWIGAIYTGNGDTSYNQKFKGKATLTADKSSSTAYMQLSSLTSEDSAVYYCNYAMDHWGQGTSVTVSS

SEQ ID NO: 36 - DNA, 339 bp, Artificial Sequence
  Description of Artificial Sequence: Synthetic GMAB 2510 Heavy Chain Nucleotide Sequence
caggagcaactgcagcagtctgagactgccctggtgaagcctggggcctcagtgaagatgtcctgcaaggcttctggcttcacatttaccaattactttatgcactgggtaaagcagacacctggacagggcctggagtggattggagctatttatacaggaaatggtgatacttcctacaatcagaagttcaaaggcaaggccaccttgactgcagacaaatcctccagcacagcctacatgcagctcagcagcctgacatctgaggactctgcggtctattactgcaactatgctatggaccactggggtcaaggaacctcagtcaccgtctcctca

SEQ ID NO: 37 - protein, 112 aa, Artificial Sequence
  Description of Artificial Sequence: Synthetic GMAB 2510 Light Chain Amino Acid Sequence
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPRTFGGGTKLEIK

SEQ ID NO: 38 - DNA, 336 bp, Artificial Sequence
  Description of Artificial Sequence: Synthetic GMAB 2510 Light Chain nucleotide sequence
gatgttgtgatgacccagactccactcactttgtcggttaccattggacaaccagcctccatctcttgcaagtcaagtcagagcctcttagatagtgatggaacgacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcggacgttcggtggaggcaccaagctggaaatcaaa

SEQ ID NO: 39 - skipped

SEQ ID NO: 40 - protein, 324 aa, Artificial Sequence; identical to SEQ ID NO: 33
  Description of Artificial Sequence: Synthetic GMA 2510 Heavy Chain Constant Region Amino acid

SEQ ID NO: 41 - protein, 107 aa, Artificial Sequence; identical to SEQ ID NO: 34
  Description of Artificial Sequence: Synthetic GMA 2510 Light Chain Constant Region Nucleotide